SEQUENCE LISTING (32 sequences)

SEQ ID NO: 1 - protein, 712 aa, Zea mays
MWAERVVGERRMRQIQRFARNAKLTVVCLLLTVVVLRGTIGAGRFGTPQQVLIELRQHFVSHPHRALAEHHDARSRASTTTTSSSSSSGRRDEPDPPPRSLRDPPYTLGPKISDWDEQRAAWHRRHPETPPFLNDIKPRVLRDGPAHLPRPTAERGISPLFPDPRELGRTNPPPTPAGRESHEVTARVRRRFPFSPHVVVMDTSHHYHPWLNFSLAHHCDLEEEERGAAAELAAIAGAAPPPKLEDFLGGGVATGGPEAVAPAEMYDSDLKFIAAAGFLGGSAAAAATSPLSSLDQAGSKLALPAAAAAPAPEQRKAVDSFGQRTSIYRGVTRTKQVFGDCTGGLAGTRHICGTTAADAKGRAARAAKKHHIADGTIFFHESEGGYDKEEKAARAYDLAALKYWGSSTTTNFPVAEYEKEVEEMKNMTRQEFVASLRRKSSGFSRGASIYRGVTRHHQHGRWQARIGRVAGNKDLYLGTFSTEEEAAEAYDIAAIKFRGLNAVTNFEISRYNVETIMSSNLPVASMSSSAAAAAGGRSSKALESPPSGSLDGGGGMPVVEASTAPPLFIPVKYDQQQQEYLSMLALQQHHQQQQAGNLLQGPLVGFGGLYSSGVNLDFANSHGTAAPSSMAHHCYANGTASASHEHQHQMQQGGENETQPQPQQSSSSCSSLPFATPVAFNGSYESSITAAGPFGYSYPNVAAFQTPIYGME

SEQ ID NO: 2 - DNA, 2139 bp, Artificial Sequence
  cDNA of SEQ ID NO 1
atgtgggcggagagggtggtcggcgagcgccggatgcggcagatccagcgcttcgcgcggaacgccaagctcaccgtcgtctgcctcctcctcaccgtagttgtgctccgcggcaccatcggcgccggtcgcttcggcacgccgcagcaggtcctcatcgagctccgccagcacttcgtctcccacccgcaccgggcgctcgcggagcaccacgacgccaggtccagggcctccaccaccaccacgtcgtcctcgtcttcctccggccgccgcgacgagcccgacccaccgccgaggtcgctcagggacccgccctacacgctggggcccaagatctccgactgggacgagcagcgcgccgcctggcaccgccgccacccggagacgccgcctttcctcaacgacatcaagccacgggtgctgcgagacggccctgctcatcttcctcgtcctaccgccgagcgcggaatctcacctctcttccccgacccgagagagctcggtcggacaaacccaccacccacaccggccggccgggagagccacgaagttactgcgcgcgtacgtcgccggtttcctttctcaccgcacgtcgtcgtcatggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacgaacgaagcaagtgtttggtgattgcaccggtggactggcaggtacgaggcacatctgtgggacaacagctgccgacgcgaagggcagagccgcaagggccgccaagaagcatcacattgctgatggtactattttttttcatgaatctgaaggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctaccgaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcggcggcggcggcggcgggtggccggagcagcaaggcgctggagtcccctccgtccggctcgcttgacggcggcggcggcatgccagtcgtcgaagccagcacggcaccgccgctgttcattccggtgaagtacgaccagcagcagcaggagtacctgtcgatgctcgcgttgcagcagcaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcctccgcctcgcatgagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatga

SEQ ID NO: 3 - protein, 485 aa, Zea mays
MDMDMSSAYPHHWLSFSLSNNYHHGLLEAFSNSSGTPLGDEQGAVEESPRTVEDFLGGVGGAGAPPQPAAAADQDHQLVCGELGSITARFLRHYPAAPAGTTVENPGAVTVAAMSSTDVAGAESDQARRPAETFGQRTSIYRGVTRHRWTGRYEAHLWDNSCRREGQSRKGRQGGYDKEEKAARAYDLAALKYWGPTTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVESILSSDLPVGGGASGRAPAKFPLDSLQPGSAAAMMLAGAAAASQATMPPSEKDYWSLLALHYQQQQEQERQFPASAYEAYGSGGVNVDFTMGTSSGNNNNNTGSGVMWGATTGAVVVGQQDSSGKQGNGYASNIPYAAAAMVSGSAGYEGSTGDNGTWVTTTTSSNTGTAPHYYNYLFGME

SEQ ID NO: 4 - DNA, 1458 bp, Artificial Sequence
  cDNA of SEQ ID NO 3
atggacatggacatgagctcagcttatccccaccattggctctccttctccctctccaacaactaccaccatggcctactcgaagccttctctaactcctccggtactcctcttggagacgagcagggcgcagtggaggagtccccgaggacggtggaggacttcctcggcggcgtcggtggcgccggcgccccgccgcagccggcggcggctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggccggggcggagtccgaccaggcgaggcggcccgccgagacgttcggccagcgcacatccatctaccgtggcgtcaccaggcaccggtggacggggagatatgaggcgcacctgtgggacaacagctgccgccgggagggccaaagccgcaaaggacggcaaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaactttgacatgagccgctacgacgtggagagcatcctcagcagcgacctccccgtcgggggcggagctagcggtcgcgcccccgccaagttcccgttggactcgctgcagccggggagcgctgccgccatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctatggtttctggatctgctggctacgagggctccaccggcgacaatggaacctgggttactacgactaccagcagcaacaccggcacggctccccactactacaactatctcttcgggatggagtag

SEQ ID NO: 5 - protein, 367 aa, Triticum aestivum
MEMQQQYFGGDGDADWFHQLALLPPLPISSSLPPLPMSEGSCLPMAAAAAAALPLGDCSSALMIRPEEQMSCLPMNPSPAVVDDVYSSYAPNNVDVLPPFPAGLDDALLMESFSDIDLEEFADAFGHKIKTEPLDDAMVPADHDFAAQAQQACPVVIMNQQQLNAPRDVRLLIDPDDDDSTVVAGGYEAAAVGCAEQKQVRPAPRRVRKSSGGARPAAGGKSLDHIGFEELRTYFYMPITKAAREMNVGLTVLKKRCRELGVARWPHRKMKSLRSLILNIQEMGKGATSPAAVQGELEALERYCAIMEENPAIELTEQTKKLRQACFKENYKRRRAAASVNLLDHCYNDLASHEQQMPLPQMGFFGF

SEQ ID NO: 6 - DNA, 1104 bp, Artificial Sequence
  cDNA of SEQ ID NO 5
atggagatgcaacagcaatacttcggcggtgatggcgatgctgactggttccaccagctcgccttgctcccgcctttgccgatctcttcgtctctgccgcctctccccatgagcgagggcagctgcttacccatggccgccgccgccgcggcggcgcttcctcttggggattgctcatcagctctcatgattaggccggaagaacagatgagctgcctgccgatgaacccttcgccagctgttgtcgatgatgtgtacagcagctacgcccccaacaatgtcgacgtcctcccgccgtttcctgcaggtctcgacgacgcgctgctcatggagtccttcagcgatatcgacctggaggagttcgccgacgccttcggccacaagattaagaccgagcctctcgacgacgctatggtgccggcggatcacgatttcgcggcgcaagcgcaacaggcgtgcccagtggtgatcatgaaccagcagcagctgaatgcaccacgcgacgtgcgcctgctcatagatcccgacgacgatgactcaactgtcgtcgccgggggctatgaggctgcggccgttgggtgcgctgagcagaagcaggtgaggccggcgccacgtcgtgtgcgcaagagcagcggtggggcacgcccagccgccggtgggaaaagcctcgatcacatagggtttgaggagctacgtacgtatttctacatgcctatcaccaaggcggcgcgggagatgaacgttggtctcaccgtgctcaagaagcgctgccgagagctcggggtcgcccgctggcctcaccggaagatgaagagcctcaggtcactcatcctcaacatccaggagatggggaagggcgcaacgtcgccggcggctgtgcaaggggaactagaggcgcttgagaggtattgcgccataatggaggagaacccggcgatcgagctgactgagcagaccaagaagctgcggcaggcctgctttaaggagaactacaagaggaggagagcggcggcctccgtcaacttgctcgaccattgctacaacgacttggccagtcatgagcagcagatgccattgccacagatgggtttctttgggttctaa

SEQ ID NO: 7 - protein, 676 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 8 - DNA, 2031 bp, Artificial Sequence
  cDNA of SEQ ID NO 7
atggcttcagcgaacaactggctgggcttctcgctctcgggccaggataacccgcagcctaaccaggacagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagggaagggcagagtcgcaagggtaggcaaggtggctatgacaaggaggacaaggcagcaagggcttacgatttggcagctctcaagtattggggcactacgacaacaacaaatttccctataagcaactacgaaaaagagctagaagaaatgaaacatatgactagacaggagtacattgcatacctaagaagaaatagcagtggattttctcgtggggcatcaaagtatcgtggagtaactagacatcatcagcatgggagatggcaagcaaggatagggagagttgcaggaaacaaggatctttacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtgggtaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 9 - protein, 706 aa, Zea mays
MATVNNWLAFSLSPQELPPSQTTDSTLISAATADHVSGDVCFNIPQDWSMRGSELSALVAEPKLEDFLGGISFSEQHHKSNCNLIPSTSSTVCYASSAASTGYHHQLYQPTSSALHFADSVMVASSAGVHDGGSMLSAAAANGVAGAASANGGGIGLSMIKNWLRSQPAPMQPRAAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAATTGLYHPYAQQPMRGGGWCKQEQDHAVIAAAHSLQDLHHLNLGAAGAHDFFSAGQQAAAAAAMHGLASIDSASLEHSTGSNSVVYNGGVGDSNGASAVGSGGGYMMPMSAAGATTTSAMVSHEQMHARAYDEAKQAAQMGYESYLVNAENNGGGRMSAWGTVVSAAAAAAASSNDNIAADVGHGGAQLFSVWNDT

SEQ ID NO: 10 - DNA, 2121 bp, Artificial Sequence
  cDNA of SEQ ID NO 9
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 11 - protein, 311 aa, Zea mays
MTGLDEALMLPFTDIDLEAFDNAEEQKPPVDQMVMMPPTVEHPAAAGTRAPIIIDGTATVGQNVGGGVVHAHQKAAMTTIEDSSCFRRGASCVDDDMAVVIHHVERRRQAGSTAVALLPPPQPSLPRPRARASGGAGERSAPAAAGKTRMDHIGFDELRKYFYMPITRAAREMNVGLTVLKKRCRELGVARWPHRKMKSLKSLMANVQEMGNGMSPVAVQHELAALETYCALMEENPWIELTDRTKRLRQACFKESYKRRKAAAGNAIETDHIVYSFGQHRRYKQQLLPPPTAGSTSADDRHGQSSRFFCY

SEQ ID NO: 12 - DNA, 936 bp, Artificial Sequence
  cDNA of SEQ ID NO 11
atgacgggcctcgacgaggcgctcatgctgccgttcaccgacatcgatcttgaggccttcgacaacgccgaagagcaaaagcctcctgtcgaccaaatggttatgatgccgccgacggttgaacaccccgccgccgccgggacgcgagccccaatcatcattgatggtacggcgaccgttggccaaaatgtaggtggtggtgtcgtccacgctcatcagaaggcggccatgacgaccatagaggactccagctgcttccgacgaggagccagctgtgtcgacgacgacatggccgtcgtcattcaccatgtcgagcgtcgtcgtcaagcaggctctaccgccgtggcgctattgccgccgccgcagccgtcactgccgcggccgcgtgcaagggcgagcggcggcgcgggcgagcggtcagctccggcggccgccgggaagacgaggatggaccacatcggcttcgacgagctgcgcaagtacttctacatgcccatcaccagggcggccagggagatgaacgtggggctcaccgtgctcaagaagcgctgccgcgagctcggcgtggcgcggtggcctcaccggaagatgaagagcctcaagtccctcatggccaacgtacaggaaatggggaacggcatgtcgccggtggctgtgcagcatgagcttgcggcgctggagacgtactgcgcgctcatggaggagaacccatggatcgagctcacggaccggacgaagaggctgcggcaggcctgcttcaaggagagctacaagcggaggaaggcggccgcaggcaacgctatcgagacggatcacattgtctacagctttggacagcatcgtcgttacaagcagcagctgctgcctccgccaactgcgggtagtaccagtgctgacgaccgccatggccagagcagccgtttcttttgctactga

SEQ ID NO: 13 - protein, 558 aa, Arabidopsis thaliana
MKNNNNKSSSSSSYDSSLSPSSSSSSHQNWLSFSLSNNNNNFNSSSNPNLTSSTSDHHHPHPSHLSLFQAFSTSPVERQDGSPGVSPSDATAVLSVYPGGPKLENFLGGGASTTTTRPMQQVQSLGGVVFSSDLQPPLHPPSAAEIYDSELKSIAASFLGNYSGGHSSEVSSVHKQQPNPLAVSEASPTPKKNVESFGQRTSIYRGVTRHRWTGRYEAHLWDNSCRREGQSRKGRQVYLGGYDKEDKAARAYDLAALKYWGPTTTTNFPISNYESELEEMKHMTRQEFVASLRRKSSGFSRGASMYRGVTRHHQHGRWQARIGRVAGNKDLYLGTFSTQEEAAEAYDIAAIKFRGLNAVTNFDISRYDVKSIASCNLPVGGLMPKPSPATAAADKTVDLSPSDSPSLTTPSLTFNVATPVNDHGGTFYHTGIPIKPDPADHYWSNIFGFQANPKAEMRPLANFGSDLHNPSPGYAIMPVMQEGENNFGGSFVGSDGYNNHSAASNPVSAIPLSSTTTMSNGNEGYGGNINWINNNISSSYQTAKSNLSVLHTPVFGLE

SEQ ID NO: 14 - DNA, 1677 bp, Artificial Sequence
  cDNA of SEQ ID NO 13
atgaagaacaataacaacaaatcttcttcttcttctagctatgattcttctttgtctccttcttcttcatcctcctcccaccagaactggctctctttctctctctccaacaataacaacaacttcaattcttcctcaaaccctaatctcacttcctccacatcagatcatcatcatcctcacccttctcacctctctctctttcaagctttctccacttctccagtcgaacggcaagatgggtcaccgggagtttcacccagcgatgccacggcggttctttccgtataccccggcggtcctaaacttgagaacttcctcggcggaggagcctcaacgacgacaacaagaccaatgcaacaagtgcaatctcttggcggcgttgtcttctcttccgacctacagccaccgcttcatcctccgtccgccgccgagatctacgactctgagctcaagtcaatagccgctagcttcctaggaaactactccggtggacactcgtcggaggtctctagcgtacataaacaacaaccgaatcctctagctgtctcagaggcttcgcctactccgaagaagaacgtagagagttttggacaacgtacctcgatttatagaggagtcacaagacatagatggactggaagatacgaagctcatctatgggataatagttgccgaagagaaggccaaagcagaaaaggaagacaagtttatttaggtggttatgataaggaagataaagcagctagagcttacgaccttgcagctcttaagtattggggtcctacaactacgactaatttcccgatatcaaattacgaatctgaacttgaagaaatgaaacacatgactcgacaagagttcgttgcttctttaagacggaaaagcagtggattctctaggggtgcctccatgtacagaggcgtcactagacatcatcagcatggtcgatggcaggcacgaattggaagagttgcaggcaacaaagacctttatcttggcacatttagcactcaagaggaagctgcagaagcttatgatatagcagcgatcaaattccgcggtctaaatgcagtcaccaatttcgacatcagtcgatatgatgtcaaatcaattgctagctgtaatctccctgtgggtggactaatgcctaaaccttctccagcaaccgcagcggctgacaaaaccgttgatctttctccatccgactctccatctctaaccacaccgtccctcacgttcaatgtggcaacaccggtcaatgaccatggaggaactttttaccacactggtataccaatcaaaccagacccggctgatcattattggtccaacatctttggattccaggcaaacccgaaagcagaaatgcgaccattagcaaactttgggtcggatcttcataacccttctcctggttatgctataatgccggtaatgcaggaaggtgaaaacaactttggtggtagttttgttgggtctgatgggtataacaatcattccgctgcatcgaacccggtctcagcaattccgctgtcctcgacaactacaatgagtaacggtaacgaagggtatggtggaaacataaactggattaataacaacatttcaagttcttaccaaactgcaaaatcaaatctctctgttttgcacacaccggtttttgggttggaatga

SEQ ID NO: 15 - protein, 498 aa, Arabidopsis thaliana
MAPPMTNCLTFSLSPMEMLKSTDQSHFSSSYDDSSTPYLIDNFYAFKEEAEIEAAAASMADSTTLSTFFDHSQTQIPKLEDFLGDSFVRYSDNQTETQDSSSLTPFYDPRHRTVAEGVTGFFSDHHQPDFKTINSGPEIFDDSTTSNIGGTHLSSHVVESSTTAKLGFNGDCTTTGGVLSLGVNNTSDQPLSCNNGERGGNSNKKKTVSKKETSDDSKKKIVETLGQRTSIYRGVTRHRWTGRYEAHLWDNSCRREGQARKGRQVYLGGYDKEDRAARAYDLAALKYWGSTATTNFPVSSYSKELEEMNHMTKQEFIASLRRKSSGFSRGASIYRGVTRHHQQGRWQARIGRVAGNKDLYLGTFATEEEAAEAYDIAAIKFRGINAVTNFEMNRYDIEAVMNSSLPVGGAAAKRHKLKLALESPSSSSSDHNLQQQQLLPSSSPSDQNPNSIPCGIPFEPSVLYYHQNFFQHYPLVSDSTIQAPMNQAEFFLWPNQSY

SEQ ID NO: 16 - DNA, 1497 bp, Artificial Sequence
  cDNA of SEQ ID NO 15
atggctcctccaatgacgaattgcttaacgttttctctgtcaccaatggagatgttgaaatcaactgatcagtctcacttctcttcttcttacgacgattcttctactccttatctcatcgataacttctatgctttcaaagaagaagctgagatagaagctgctgctgcttcaatggcggattcaacaaccttatctacttttttcgatcattctcagactcagattccaaagctggaagatttcctcggtgattcctttgtccgttactctgataaccaaacagagacccaagactcttcttctctcactccattctacgatccacgtcaccgcaccgttgccgaaggagttacagggttcttctctgatcatcatcagccagatttcaagacgataaactcgggaccagaaatcttcgatgactcaacaacttccaacatcggtggtactcatctctccagtcacgtggtggagtcatcaacgacggcgaagttagggtttaacggtgattgcaccaccaccggaggagttttgtctctaggggttaacaacacatcagatcaacctttgagctgtaacaatggcgagagaggtggaaacagtaacaagaagaaaacagtttctaagaaggaaacatcagatgattcaaagaagaagattgtcgaaacattgggacaaagaacttcaatttatcgtggagtcacccgacatagatggactggaagatacgaagcgcatctatgggataacagctgtaggagggaaggtcaagccagaaaaggacgtcaagtgtacttaggtggatatgacaaggaagatagagcagctagagcctatgacttggcagctttaaaatactggggttctactgctactacaaattttccggtctcgagttattcaaaagaacttgaggaaatgaatcacatgaccaagcaagagtttattgcatctcttaggaggaaaagtagcggtttttcgagaggagcttcaatatatagaggtgtcacaaggcatcatcaacaaggtcgctggcaagcaagaatcggccgtgtcgcaggaaacaaagatctttacctcggaacctttgcaaccgaagaggaagcagcagaggcttatgacattgcagccataaagttcagaggaatcaacgcagtaactaactttgagatgaacaggtatgacattgaagctgtcatgaatagttctttacctgtaggaggagcagctgcgaaacgccacaaactcaaactcgctcttgaatctccttcttcatcatcctctgaccataacctccaacaacaacagttgcttccgtcctcttctccctcggatcaaaaccctaactcaatcccatgtggcattccatttgagccttcagttctctattaccaccagaacttctttcagcattatcctttggtctctgactctacaattcaagctcctatgaaccaagctgagtttttcttgtggcctaaccagtcttactaa

SEQ ID NO: 17 - protein, 256 aa, Arabidopsis thaliana
MSSSKHSSVFNYSALFLSLFLQQMDQNSLHHLDSPKIENEYEPDSLYDMLDKLPPLDSLLDMEDLKPNAGLHFQFHYNSFEDFFENIEVDNTIPSDIHLLTQEPYFSSDSSSSSPLAIQNDGLISNVKVEKVTVKKKRNLKKKRQDKLEMSEIKQFFDRPIMKAAKELNVGLTVLKKRCRELGIYRWPHRKLKSLNSLIKNLKNVGMEEEVKNLEEHRFLIEQEPDAELSDGTKKLRQACFKANYKRRKSLGDDYY

SEQ ID NO: 18 - DNA, 771 bp, Artificial Sequence
  cDNA of SEQ ID NO 17
atgagttcgtcaaaacattcctctgtttttaactattctgctctgtttctatcactgtttcttcaacaaatggatcagaactctcttcatcatctcgattctccaaaaatcgaaaacgagtatgaaccagattcgttatacgacatgttagataagttgcctccgcttgattctctcctagatatggaagatttgaaaccaaatgcagggttgcactttcagttccattacaatagctttgaagatttcttcgaaaacattgaagtggataacacaattccatctgatattcacttgttgacacaagagccctacttctcaagtgactcctcttcctcttcaccattggctatccaaaacgacggtctcatttccaacgtgaaagttgaaaaggtaacagttaagaagaagaggaaccttaagaaaaagaggcaagacaaattggagatgtctgagatcaaacaatttttcgataggccgatcatgaaagcggctaaagaactgaacgtgggactcactgtgttgaagaagcgatgcagggaattaggaatttaccggtggcctcaccggaagctcaagagtctaaactctcttataaagaatctcaagaatgttggaatggaagaggaagtgaagaacttggaggaacataggtttcttattgaacaagaacctgatgcagaactcagtgatggaaccaagaagctaaggcaagcttgtttcaaagccaattataagagaagaaaatcacttggtgatgattattattga

SEQ ID NO: 19 - protein, 574 aa, Arabidopsis thaliana
MNSNNWLGFPLSPNNSSLPPHEYNLGLVSDHMDNPFQTQEWNMINPHGGGGDEGGEVPKVADFLGVSKPDENQSNHLVAYNDSDYYFHTNSLMPSVQSNDVVVAACDSNTPNNSSYHELQESAHNLQSLTLSMGTTAGNNVVDKASPSETTGDNASGGALAVVETATPRRALDTFGQRTSIYRGVTRHRWTGRYEAHLWDNSCRREGQSRKGRQVYLGGYDKEDKAARSYDLAALKYWGPSTTTNFPITNYEKEVEEMKHMTRQEFVAAIRRKSSGFSRGASMYRGVTRHHQHGRWQARIGRVAGNKDLYLGTFSTEEEAAEAYDIAAIKFRGLNAVTNFEINRYDVKAILESSTLPIGGGAAKRLKEAQALESSRKREAEMIALGSSFQYGGGSSTGSGSTSSRLQLQPYPLSIQQPLEPFLSLQNNDISHYNNNNAHDSSSFNHHSYIQTQLHLHQQTNNYLQQQSSQNSQQLYNAYLHSNPALLHGLVSTSIVDNNNNNGGSSGSYNTAAFLGNHGIGIGSSSTVGSTEEFPTVKTDYDMPSSDGTGGYSGWTSESVQGSNPGGVFTMWNE

SEQ ID NO: 20 - DNA, 1725 bp, Artificial Sequence
  cDNA of SEQ ID NO 19
atgaattctaacaactggcttggctttcctctttcaccgaacaactcttctttgcctcctcatgaatacaaccttggcttggtcagcgaccatatggacaacccttttcaaacacaagagtggaatatgatcaatccacacggtggaggaggagatgaaggaggagaggttccaaaagtggccgattttctcggtgtgagcaaaccggacgaaaaccaatccaaccacctagtagcttacaacgactcagactactacttccataccaatagcttgatgcctagcgtccaatcaaacgatgtcgttgtagcagcttgtgactccaatactcctaacaacagtagctatcatgagcttcaagagagtgctcacaatctacagtcacttactttgtccatggggaccaccgctggtaataatgttgtagacaaagcttcaccatccgagaccaccggggataacgctagcggtggagcactagccgttgttgagacggccacgccaagacgtgcattggacactttcggacaacgaacctcgatctatcgtggtgtcacaagacatcgatggactggtcgatatgaggctcatctatgggataatagttgtagaagggaaggccagtctaggaaaggaagacaagtttacttgggtggatatgacaaagaagataaagcagcaagatcatatgatctagctgcacttaagtactggggtccttcaactactactaatttccccattacaaactacgagaaagaagtagaggaaatgaagcacatgacgagacaagagttcgtggctgccattagaaggaaaagtagtggattttcgagaggcgcttcgatgtatcgaggagttacaaggcatcaccaacatggaagatggcaagcaaggatcggccgagtcgccggaaacaaagacctctacttgggaacttttagcactgaggaagaagcagcagaagcttacgatatagctgcaataaagtttagaggacttaatgcagtgaccaacttcgagatcaaccggtacgacgtgaaagccattctagagagtagcactcttcccatcggaggaggcgcagctaaacggctcaaagaagctcaagctcttgagtcttcaaggaaacgcgaggcggagatgatagcccttggttcaagtttccagtacggtggtggctcgagcacaggctctggctccacctcatcaagacttcagcttcaaccttaccctctaagcattcaacaaccattagagccttttctatctcttcagaacaatgacatctctcattacaacaacaacaatgctcacgattcctcctcttttaatcaccatagctatatccagacacaacttcatctccaccaacagaccaacaattacttgcagcaacagtcgagccagaactctcagcagctctacaatgcgtatcttcatagcaatccggctctgcttcatggacttgtctctacctctatcgttgacaacaataataacaatggaggctctagtgggagctacaacactgcagcatttcttgggaaccacggtattggtattgggtccagctcgactgttggatcgaccgaggagtttccaaccgttaaaacagattacgatatgccttccagtgatggaaccggagggtatagtggttggaccagtgagtctgttcaggggtcaaaccctggtggtgttttcactatgtggaatgagtaa

SEQ ID NO: 21 - protein, 568 aa, Arabidopsis thaliana
MNSNNWLAFPLSPTHSSLPPHIHSSQNSHFNLGLVNDNIDNPFQNQGWNMINPHGGGGEGGEVPKVADFLGVSKSGDHHTDHNLVPYNDIHQTNASDYYFQTNSLLPTVVTCASNAPNNYELQESAHNLQSLTLSMGSTGAAAAEVATVKASPAETSADNSSSTTNTSGGAIVEATPRRTLETFGQRTSIYRGVTRHRWTGRYEAHLWDNSCRREGQSRKGRQVYLGGYDKEEKAARAYDLAALKYWGPSTTTNFPITNYEKEVEEMKNMTRQEFVASIRRKSSGFSRGASMYRGVTRHHQHGRWQARIGRVAGNKDLYLGTFSTEEEAAEAYDIAAIKFRGLNAVTNFEINRYDVKAILESNTLPIGGGAAKRLKEAQALESSRKREEMIALGSNFHQYGAASGSSSVASSSRLQLQPYPLSIQQPFEHLHHHQPLLTLQNNNDISQYHDSFSYIQTQLHLHQQQTNNYLQSSSHTSQLYNAYLQSNPGLLHGFVSDNNNTSGFLGNNGIGIGSSSTVGSSAEEEFPAVKVDYDMPPSGGATGYGGWNSGESAQGSNPGGVFTMWNE

SEQ ID NO: 22 - DNA, 1707 bp, Artificial Sequence
  cDNA of SEQ ID NO 21
atgaattctaacaactggctcgcgttccctctatcaccaactcactcttctttgccgcctcacattcactcttcacaaaattctcatttcaatctaggtttggtcaacgacaatatcgacaacccttttcaaaaccaaggatggaatatgatcaatccacatggtggaggcggcgaaggtggagaggttccaaaagtggctgatttcttaggagtgagcaaatcgggggatcatcacaccgatcacaacctcgtaccttataacgacattcatcaaaccaacgcctccgactactactttcaaaccaatagcttgttacctacagtcgtcacttgtgcctctaatgctcctaataattatgagcttcaagagagtgcacacaatttgcaatctctcactctctctatgggaagtactggagctgccgctgcagaagtcgccactgtgaaagcctcgccggctgagactagtgccgataatagtagcagcactaccaacacaagtggaggagccatcgttgaggctacaccgagacggactttggaaacttttggacaacgaacctctatctatcgtggagttacaagacatagatggaccggtagatatgaagctcatctttgggataatagctgtagaagagaaggacaatcaaggaaaggaagacaagtctacttaggtgggtatgacaaagaagagaaagcagccagagcatatgatctagctgcacttaaatattggggtccctctactactaccaactttccgataactaactacgagaaggaagtagaggagatgaaaaacatgacgagacaagagtttgtggcttctataagaaggaaaagtagcggattctcgcgtggtgcatccatgtatcgtggagtaacaaggcatcatcaacatggaagatggcaagcaaggatcggccgagttgctggaaacaaagatctctacttgggaacattcagcacggaggaagaagcagcagaagcttatgacatagctgcgataaagtttcgaggtctaaacgcggttacaaactttgagataaatcggtatgatgtgaaagccatcctggagagcaacacacttcctataggaggtggtgcggctaaacggctcaaagaagctcaagctctagaatcatcaagaaaacgagaggaaatgatagccctcggatcaaatttccatcaatatggtgcagcgagcggctcgagctctgttgcttccagctctaggcttcagcttcaaccttaccctctaagcattcaacaaccttttgagcatcttcatcatcatcagcctttacttactctacagaacaacaacgatatctctcagtatcatgattcctttagttacattcagacgcagcttcatcttcaccaacaacaaaccaacaattacttgcagtcttctagtcacacttcacagctctacaatgcttatcttcagagtaaccctggtctgcttcatggatttgtctctgataataacaacacttcagggtttcttggaaacaatgggattggtattgggtcaagctctaccgttggatcatcggctgaggaagagtttccagccgtgaaagtcgattacgatatgcctccttccggtggagctacagggtatggaggatggaatagtggagagtctgctcaaggatcgaatccaggaggtgttttcacgatgtggaatgaataa

SEQ ID NO: 23 - protein, 584 aa, Arabidopsis thaliana
MMAPMTNWLTFSLSPMEMLRSSDQSQFVSYDASSAASSSPYLLDNFYGWSNQKPQEFFKEEAQLAAAASMADSTILTTFVDPQSHHSQNHIPKLEDFLGDSSSIVRYSDNSQTDTQDSSLTQIYDPRHHHNQTGFYSDHHDFKTMAGFQSAFSTNSGSEVDDSASIGRTHLAGDYLGHVVESSGPELGFHGGSTGALSLGVNVNNNTNHRNDNDNHYRGNNNGERINNNNNNDNEKTDSEKEKAVVAVETSDCSNKKIADTFGQRTSIYRGVTRHRWTGRYEAHLWDNSCRREGQARKGRQVYLGGYDKEDKAARAYDLAALKYWNATATTNFPITNYSKEVEEMKHMTKQEFIASLRRKSSGFSRGASIYRGVTRHHQQGRWQARIGRVAGNKDLYLGTFATEEEAAEAYDIAAIKFRGINAVTNFEMNRYDVEAIMKSALPIGGAAKRLKLSLEAAASSEQKPILGHHQLHHFQQQQQQQQLQLQSSPNHSSINFALCPNSAVQSQQIIPCGIPFEAAALYHHHQQQQQHQQQQQQQNFFQHFPANAASDSTGSNNNSNVQGTMGLMAPNPAEFFLWPNQSY

SEQ ID NO: 24 - DNA, 1755 bp, Artificial Sequence
  cDNA of SEQ ID NO 23
atgatggctccgatgacgaactggttaacgttttctctgtcaccaatggagatgttgaggtcatctgatcagtctcagtttgtctcctatgacgcttcttccgccgcttcctcctctccttatctcctcgataatttctatggttggtcaaaccaaaaacctcaggagtttttcaaagaagaagctcagttagcagcagcagcttcaatggcggattcaacaatcttaacaacattcgtagacccacaatctcatcattctcagaatcacatcccaaagctcgaagattttctcggtgactcttcttctatcgttcgttactccgacaacagtcaaaccgacacacaagactcttccctcactcaaatctacgatccacgtcaccaccataaccaaaccggcttttactccgatcaccacgatttcaaaaccatggccggttttcaatccgctttctctactaactccggttcagaggtcgatgactctgcttctatcggaaggactcatcttgctggagactatttgggacacgtggttgaatcttctggtccggagctagggtttcacggtggatctaccggagctttgtcacttggtgttaacgtcaataacaatactaatcaccggaatgataatgataatcattaccgaggcaataacaatggtgagagaatcaacaacaacaacaacaatgacaacgagaagacagattctgagaaggagaaggctgttgtggctgtggaaacatcagattgttctaataagaagattgctgatacgtttggtcaaaggacttcgatttacagaggtgttactcgacatagatggacgggaagatatgaagcacatctatgggataatagctgtaggagagaaggtcaggccaggaagggacgtcaagtatacttgggtggatatgacaaagaagataaggcagctcgagcttacgatttagcagctctgaaatactggaatgctactgctaccaccaatttccctattacgaattactcgaaagaagtggaggaaatgaagcacatgaccaagcaagagttcattgcctccctcaggaggaagagtagcggtttctctagaggagcttcgatataccgaggtgttacaaggcatcatcaacaaggacgttggcaagcaaggattggccgagttgctgggaacaaagatctttaccttggaacctttgcaacggaagaggaagcagctgaagcgtatgacatagcagcaatcaaattcagaggaataaacgctgtaactaactttgagatgaaccgttacgacgttgaagccatcatgaagagtgcacttcccatcggtggtgcagctaaacgtcttaagctctctttggaagctgctgcttcatcagagcagaaaccaatcctcggtcatcatcaacttcaccatttccagcaacaacaacaacaacaacagcttcagcttcagtcatctcctaatcacagtagcattaacttcgctctctgtcctaattcagcagttcagtctcaacagatcattccttgtggaatcccttttgaagcagctgctctttaccaccaccaccaacaacaacagcaacaccaacagcagcagcagcaacagaacttcttccagcattttccggcgaatgcagcttctgactcgaccgggtctaacaacaactccaacgttcagggaacaatgggacttatggcaccaaatccggctgagttcttcctctggcctaatcagtcttactaa

SEQ ID NO: 25 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNNINNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGTTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 26 - DNA, 1755 bp, Artificial Sequence
  cDNA of SEQ ID NO 25
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaataacattaacaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggaaccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaataatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 27 - protein, 374 aa, Triticum aestivum
MEMQQYFGGCGDGDADWFHQLALLPPLPVSSSLPPLPMSEGSCLPMAAAAPTLPLGDCSSALMIRPEEQMGCLQMIPPQAVADDEYSSYATNNVDVLPPFPAGLDDPTAGLDDALLMESFRDIDLEEFADAVGPKIKTEPLDDAMVPADHDFAAQVQQARPVVIMNQQQLNAPHGVRLLNDPDDDDSAVVAGGYEAAAVGCAEQKRVRPAPRRVRKSSGGSRPAAGGKSLDHIGFEELRTYFYMPITKAAREMNVGLTVLKKRCRELGVARWPHRKMKSLRSLILNIQDMGKGATSPAAVQGELEALERYCAIMEENPAIELTEQTKKLRQACFKENYKRRRAAASVNLLEHCYNDLGSHEQQMPLPQMGFFGF

SEQ ID NO: 28 - DNA, 1125 bp, Artificial Sequence
  cDNA of SEQ ID NO 27
atggagatgcagcagtacttcggcggctgcggcgatggcgatgctgactggttccatcagctcgccttgctcccgcctttgccggtctcttcgtctctgccgcctctccccatgagcgagggcagctgcttacctatggccgccgccgccccaacgcttcctcttggggattgctcatcagctctcatgattaggccggaagaacagatgggctgcctgcagatgatacctccacaggctgttgccgatgatgagtacagcagctacgccaccaacaatgtcgacgtcctcccgccgtttcctgcaggtctcgatgatcccacggcaggcctcgacgacgcgctgctcatggagtccttcagagacatcgacctggaggagttcgccgacgccgtcggccccaagattaagaccgagcctctcgacgacgccatggtgccggcggatcacgatttcgcggcgcaagtgcaacaggcgcgccccgtggtgatcatgaaccagcagcagctgaatgcgccacacggcgtgcgcctgctcaatgatcccgacgacgatgactcagctgtcgtcgccgggggctatgaggcggcggccgttgggtgcgctgagcagaagcgggtgaggccggcgccacgtcgtgtgcggaagagcagcggtgggtcacgccctgccgccggtgggaaaagcctcgatcacatagggtttgaggagctgcgtacgtatttctacatgcctatcaccaaggcggcgcgggagatgaacgtcggtctcaccgtgctcaagaagcgctgccgtgagctcggtgtcgcccgttggcctcaccggaagatgaagagcctcaggtctctcatccttaacatccaggacatggggaagggcgccacgtcgccggcggcggtgcaaggggagctggaggcgcttgagaggtattgtgccataatggaggagaacccggcgatcgagctgacggagcagaccaagaagctgaggcaggcctgctttaaggagaactacaagaggaggagagcggcggcctccgtcaacttgctcgagcattgctacaacgacttgggcagtcatgagcagcagatgccattgccacagatgggtttctttgggttctaa

SEQ ID NO: 29 - protein, 298 aa, Arabidopsis thaliana
MADHTTKEQKSFSFLAHSPSFDHSSLSYPLFDWEEDLLALQENSGSQAFPFTTTSLPLPDLEPLSEDVLNSYSSASWNETEQNRGDGASSEKKRENGTVKETTKKRKINERHREHSVRIISDITTYTTSSAPTTLSKETVSRYFYMPITQAAIALNVGLTLLKRRCRELGIRRWPHRKLMSLNTLISNVKELQKMEGEENAEKLQDALEMLEKEKRTIEDLPDLEFKDKTKRLRQACFKANHKRKKKRSLKSDQSQVPSCSSSGSVPSDESVDEAGMESDEEMKYLLCGFSSEFTSGL

SEQ ID NO: 30 - DNA, 897 bp, Artificial Sequence
  cDNA of SEQ ID NO 29
atggctgatcacacaaccaaagaacagaagtcattctcattcctagctcattctccatcctttgatcacagctccttaagttatcctttattcgactgggaagaagatcttcttgctctccaagaaaactctggctctcaagcatttccttttactacaacttctctgcctttacctgatcttgaacccttgtctgaagatgtactcaattcatacagctctgcgtcatggaacgaaacagagcaaaacagaggagatggcgcttcatcggagaagaagagggaaaatggaacagtgaaagagacaactaagaagaggaaaatcaatgagagacacagagaacatagcgtgagaatcatcagcgatattactacctacacaactagttcagctccaacgacattgtcaaaggaaactgtctctcgctacttctacatgcccataactcaggctgcaatagcacttaacgttggtttaactctactaaaaaggagatgtcgcgaattgggtattcgccgatggcctcatcgtaaacttatgagcttaaacactttgatcagtaacgtcaaggagctgcagaagatggaaggcgaagagaatgcagaaaaactgcaggacgcgttggagatgcttgagaaggagaagaggacaattgaggatttgccggatttggagtttaaggacaagacaaagaggctaagacaagcttgtttcaaggctaaccacaagaggaagaagaagagaagtctcaagtccgatcagtctcaagtaccctcgtgttcaagcagcggatcagttcctagtgatgagtcggttgatgaagcaggaatggagagtgatgaagaaatgaagtatctcttgtgtggtttctcaagtgaatttactagtggtttgtga

SEQ ID NO: 31 - protein, 394 aa, Zea mays
MAMVPCGDDTDWCHVLDNFNLLLCSSSCSPNAMANRAEDCLPISAAPPGPGHHQSCCKNEVVLEASCDGAFAAADCLSSALTNLQREDDSFYLPMYSAPPAVGDEYFSDLLAPDADGIDEALLMPFSDIDLQVFDSDDEHRPPVDQMVNMIPPAVLHHPSTAGTQNGGAVHAHQKAMAVIDDSCFRRGASGVEMAVVRHHGEPRQGSSSVAPVPPPSLPGTRARRSDGRSARAGKTTKLDYIGFDELRKYFCMPITRAAREMNVGLTVLKKRCRELGVARWPHRKMKSLKSLMANVQEMGNVMSSVAVQQELAALETYCTLMEDNPWIELTDRTKKLRQACFKERYKRRRAAEVNVMDMDRIYCFGQHHHQQLLPPTTSSSDDRHGQCSRSFGY

SEQ ID NO: 32 - DNA, 1185 bp, Artificial Sequence
  cDNA of SEQ ID NO 31
atggcgatggtgccatgcggcgatgacaccgactggtgccacgtgctggacaacttcaacctgttgctgtgttcgtcgtcctgctcgccgaatgctatggccaacagagcggaagactgtctgccgatatctgctgctccacccggacccggccatcatcagagctgctgcaaaaacgaagtcgtcctcgaagcctcttgtgatggcgcgtttgctgcagccgactgcttgtcttcggctctgacgaacctgcagagggaggacgacagtttctatttgcccatgtactctgcgccacccgcagtcggcgatgagtacttctccgatctactcgcgcccgatgccgacggcattgacgaggcgctcctgatgccgttcagcgacatcgatcttcaggtcttcgacagtgacgacgagcacaggcctcctgtcgaccaaatggttaatatgatcccgccggcggttcttcatcatccctccaccgccgggacgcaaaatggaggtgccgttcatgctcatcagaaggccatggcggtcatcgatgactcctgtttccgacgaggagccagtggtgtcgagatggccgtcgtcaggcatcatggtgagcctcgtcaaggaagctcttccgtggcgccagtgccgccaccgtcactgccggggacgcgtgcaaggaggagcgacggccgatcagctcgggcggggaagacgacgaagctggactacatcggcttcgacgagctgcggaagtacttctgcatgcccatcaccagggcggcgagggagatgaacgtcgggctcaccgtgctcaagaagcgctgccgcgagctcggcgtggcgcggtggcctcaccggaagatgaagagcctcaagtccctcatggccaacgtccaggaaatggggaacgtcatgtcctcggtggctgtgcagcaggagcttgcggcgctcgagacgtactgcacgctcatggaggacaatccctggatcgagctcacggacaggaccaagaagctgcgccaggcgtgcttcaaggagaggtacaagcgtaggagggcggccgaagtcaacgtcatggatatggatcgcatctactgctttggccagcatcaccaccagcagctgctgcctccgacgacaagcagttctgacgaccgccatggccagtgcagccgttcctttggctactga

(19) United States
(12) Patent Application Publication
(10) Pub. No.: US 2021/0254087 A1
(43) Pub. Date: Aug. 19, 2021

(54) METHODS FOR ENHANCING GENOME ENGINEERING EFFICIENCY

(71) Applicant: KWS SAAT SE & CO. KGaA, Einbeck (DE)

(72) Inventor: Ling MENG, St. Louis, MO (US)

(73) Assignee: KWS SAAT SE & CO. KGaA, Einbeck (DE)

(21) Appl. No.: 17/251,596

(22) PCT Filed: Jun. 14, 2019
(86) PCT No.: PCT/EP2019/065643

§ 371 Date: Dec. 11, 2020

Related U.S. Application Data
(60) Provisional application No. 62/685,626, filed on Jun. 15, 2018.

(51) Int. Cl. C12N 15/82

(52) U.S. Cl. CPC C12N 15/8238

(57) ABSTRACT

This document relates to methods and materials for genome engineering in eukaryotic cells, and particularly to methods for increasing genome engineering (i.e. transformation or genome editing) efficiency via co-delivery of one or more chemicals, such as protein deacetylase inhibitors, phytohormones and/or regeneration boost genes, with genome engineering components.

### TECHNICAL FIELD

This document relates to methods and materials for genome engineering in eukaryotic cells, and particularly to methods for increasing genome engineering (i.e. transformation or genome editing) efficiency via co-delivery of one or more chemicals, such as epigenetically regulating chemicals, phytohormones and/or regeneration boost genes, with genome engineering components.

### BACKGROUND OF THE INVENTION

Traditional breeding has resulted in the domesticated plants and animals, while modern biotechnology in particular genome engineering is expanding breeding capability and enabling the improvements that are not possible with only traditional crossing of close species alone. Using biotechnology various traits, such as, high-yield, herbicide tolerance and pest resistance, have been introduced into crops, which is dramatically advancing the global agriculture and food security. With foreign DNA present in product, biotechnology has however triggered biosafety and environmental concerns.

By segregating out the integrated DNA, genome-editing technology can be used to generate site-specific modification of the target genome without the presence of foreign DNA in the end plants. Moreover, by transient expression, genome editing simply involves transient editing activity to create site-specific modification without DNA integration at any points of process. The genome-edited plants, especially those derived from the transient activity, are significantly different from the conventional genome modified plants, and may not be regulated as genetically modified (GM) plants. Genome editing techniques, especially via transient editing approach, provide a highly accurate, safe and powerful plant breeding and development tool in agriculture.

Genome engineering based on transient activity however faces more challenges. Compared with stable transformation, transient engineering generally results in less modified cells, and without an integrated selectable marker, it is highly challenging to identify the engineered cells and achieve homogenous modification in the regenerated plants. These challenges hurdle the routine implementation of transient gene editing as a breeding tool for plant improvement. Novel methods and materials that enhance genome engineering efficiency are thus highly desirable.

### SUMMARY

In the present invention it was surprisingly found, that genome engineering efficiency in plant cells can be improved by co-delivery of genome engineering components with a second compound selected from the group consisting of epigenetically regulating chemicals, e.g. protein deacetylase inhibitors or DNA methyltransferase inhibitors, in particular histone deacetylase inhibitors (HDACIs, e.g. trichostatin A (TSA)), phytohormones (e.g. auxins, cytokinins) and/or proteins causing improved plant regeneration from somatic tissue, callus tissue or embryonic tissue into the cells. In addition, the co-delivery of promoting chemicals with genome engineering components offers growth benefit specifically to the transformed cells, and thus serves as a positive selection strategy for the recovery of transformed cells.

Thus, a first aspect of the present invention is a method for genetic modification in a plant cell comprising
a) co-introducing into the plant cell
   (i) a genome engineering component and
   (ii) a second compound comprising
      (ii.1) an epigenetically regulating chemical or an active derivative thereof, in particular a DNA methyltransferase inhibitor or a protein deacetylase inhibitor, preferably a histone deacetylase inhibitor (HDACi), and/or
      (ii.2) a phytohormone or an active derivative thereof, and/or
      (ii.3) a protein causing improved plant regeneration from a somatic cell, a callus cell or an embryonic cell or an expression cassette comprising a nucleic acid encoding said protein, and
b) cultivating the plant cell under conditions allowing the genetic modification of the genome of said plant cell by activity of the genome engineering component (i) in the presence of the second compound,

preferably wherein the genome engineering component (i) and/or the second compound (ii) is transiently active and/or transiently present in the plant cell.

It was found, that all three types of compounds (ii.1), (ii.2) and (ii.3) are independently capable of increasing the efficiency of the genetic modification of the plant cell effected by the genome engineering component (i). Compounds (ii.1), (ii.2) and (ii.3) can be used either alone or as a combination of two or more compounds or types of compounds, e.g. two or more compounds of type (ii.1) or one compound of type (ii.1) and one compound of type (ii.2), etc. When referring to all three types of compounds (ii.1), (ii.2) and (ii.3), the term “compound (ii)” is used synonymously.

The method for genetic modification in a plant cell may comprise a further step c) obtaining and/or selecting the genetically modified plant cell or a plant or part thereof which comprises the genetically modified plant cell or which is derived from the genetically modified plant cell and comprises the genetic modification of the genome in at least one cell. Selection may be carried out on by means of detection of the genetic modification, e.g. by means of PCR-based methods, or by means of a phenotypical characteristic, e.g. herbicide resistance, color or fluorescence marker, morphological characteristic like plant height et cetera. Such phenotypical characteristic may be conferred by an exogenous (marker) gene, stably integrating into the genome of the plant cell.

In a particular preferred embodiment, the above method does not comprise a selection process based on an exogenous selectable marker gene stably integrating into the genome of the plant cell.

In another particular preferred embodiment, one or more proteins causing improved plant regeneration from a somatic cell, a callus cell or an embryonic cell or expression cassette(s) comprising a nucleic acid encoding said one or more proteins are co-introduced. “One or more” may be at least one, at least two, at least three, at least four, or may be one, two, three, four or more. Preferably, the one or proteins have an additive effect or even synergistic effect with respect to the improved plant regeneration.

Suitable Plant Cells

Plant cells for use in the present invention can be part of or derived from any type of plant material, preferably shoot, hypocotyl, cotyledon, stem, leave, petiole, root, embryo, callus, flower, gametophyte or part thereof. It is possible to use isolated plant cells as well as plant material, i.e. whole plants or parts of plants containing the plant cells.

A part or parts of plants may be attached to or separated from a whole intact plant. Such parts of a plant include, but are not limited to, organs, tissues, and cells of a plant, and preferably seeds.

The present invention is applicable to any plant species, whether monocot or dicot. Preferably, plants which may be subject to the methods and uses of the present invention are plants of the genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, or Helianthus. More preferably, the plant is a plant of the species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, Triticum aestivum, Hordeum vulgare, Secale cereale, Helianthus annuus, Solanum tuberosum, Sorghum bicolor, Brassica rapa, Brassica napus, Brassica juncacea, Brassica oleracea, Raphanus sativus, Oryza sativa, Glycine max, and/or Gossypium sp.

Genome Engineering Component

The term “genome engineering” as used herein refers to methodologies for genetic modification in plants, i.e. for modifying the genome of a plant. Preferably the term refers to a) transformation, preferably stabile transformation, of plants or plant cells and to b) genome editing of plants or plant cells. Genome engineering may be conducted in isolated plant cells or plant tissues preferably in cell culture or in intact plants, i.e. it may be performed in vitro or in vivo.

The genome engineering component (i) can be introduced as a protein and/or as a nucleic acid encoding the genome engineering component, in particular as DNA such as plasmid DNA, RNA, mRNA or RNP.

Genome engineering can be used for the manufacture of transgenic plant material. The term “transgenic” as used according to the present disclosure refers to a plant, plant cell, tissue, organ or material which comprises a gene or a genetic construct, comprising a “transgene” that has been transferred into the plant, the plant cell, tissue organ or material by natural means or by means of transformation techniques from another organism. The term “transgene” comprises a nucleic acid sequence, including DNA or RNA, or an amino acid sequence, or a combination or mixture thereof. Therefore, the term “transgene” is not restricted to a sequence commonly identified as “gene”, i.e. a sequence encoding protein. It can also refer, for example, to a non-protein encoding DNA or RNA sequence. Therefore, the term “transgenic” generally implies that the respective nucleic acid or amino acid sequence is not naturally present in the respective target cell, including a plant, plant cell, tissue, organ or material. The terms “transgene” or “transgenic” as used herein thus refer to a nucleic acid sequence or an amino acid sequence that is taken from the genome of one organism, or produced synthetically, and which is then introduced into another organism, in a transient or a stable way, by artificial techniques of molecular biology, genetics and the like. A “plant material” as used herein refers to any material which can be obtained from a plant during any developmental stage. The plant material can be obtained either in planta or from an in vitro culture of the plant or a plant tissue or organ thereof. The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g. a protoplast, derived from at least one plant cell comprised by the plant material. The term therefore also comprises meristematic cells or a meristematic tissue of a plant.

For plant cells to be modified, despite transformation methods based on biological approaches, like Agrobacterium transformation or viral vector mediated plant transformation, and methods based on physical delivery methods, like particle bombardment or microinjection, have evolved as prominent techniques for introducing genetic material into a plant cell or tissue of interest. Helenius et al. (“Gene delivery into intact plants using the HeliosTM Gene Gun”, Plant Molecular Biology Reporter, 2000, 18 (3):287-288) discloses a particle bombardment as physical method for introducing material into a plant cell. Currently, there thus exists a variety of plant transformation methods to introduce genetic material in the form of a genetic construct into a plant cell of interest, comprising biological and physical means known to the skilled person on the field of plant biotechnology and which can be applied to introduce at least one gene encoding at least one wall-associated kinase into at least one cell of at least one of a plant cell, tissue, organ, or whole plant. Notably, said delivery methods for transformation and transfection can be applied to introduce the tools of the present invention simultaneously. A common biological means is transformation with Agrobacterium spp. which has been used for decades for a variety of different plant materials. Viral vector mediated plant transformation represents a further strategy for introducing genetic material into a cell of interest. Physical means finding application in plant biology are particle bombardment, also named biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran or polyethylenimine, or combinations thereof. The above delivery techniques, alone or in combination, can be used for in vivo (including in planta) or in vitro approaches.

The term “genome editing” as used herein refers to strategies and techniques for the targeted, specific modification of any genetic information or genome of a plant cell. As such, the terms comprise gene editing, but also the editing of regions other than gene encoding regions of a genome, such as intronic sequences, non-coding RNAs, miRNAs, sequences of regulatory elements like promoter, terminator, transcription activator binding sites, cis or trans acting elements. Additionally, the terms may comprise base editing for targeted replacement of single nucleobases. It can further comprise the editing of the nuclear genomeas well as other genetic information of a plant cell, i.e. mitochondrial genome or chloroplast genome as well as miRNA, pre-mRNA or mRNA. Furthermore, the terms “genome editing” may comprise an epigenetic editing or engineering, i.e., the targeted modification of, e.g., DNA methylation or histone modification, such as histone acetylation, histone methylation, histone ubiquitination, histone phosphorylation, histone sumoylation, histone ribosylation or histone citrullination, possibly causing heritable changes in gene expression.

According to preferred embodiments of the invention, the genome engineering component comprises
a) a double-stranded DNA break (DSB) inducing enzyme or a nucleic acid encoding same, which preferably recognizes a predetermined site in the genome of said cell, and optionally a repair nucleic acid molecule, or
b) a single-stranded DNA or RNA break (SSB) inducing enzyme or a nucleic acid encoding same, which preferably recognizes a predetermined site in the genome of said cell, and optionally a repair nucleic acid molecule, or
c) a base editor enzyme, optionally fused to a disarmed DSB or SSB inducing enzyme, which preferably recognizes a predetermined site in the genome of said cell, or
d) an enzyme effecting DNA methylation, histone acetylation, histone methylation, histone ubiquitination, histone phosphorylation, histone sumoylation, histone ribosylation or histone citrullination, optionally fused to a disarmed DSB or SSB inducing enzyme, which preferably recognizes a predetermined site in the genome of said cell.

As used herein, a “double-stranded DNA break inducing enzyme” or “DSBI enzyme” is an enzyme capable of inducing a double-stranded DNA break at a particular nucleotide sequence, called the “recognition site” or “predetermined site”. Accordingly, a “single-stranded DNA or RNA break inducing enzyme” or “SSBI enzyme” is an enzyme capable of inducing a single-stranded DNA or RNA break at a particular nucleotide sequence, called the “recognition site” or “predetermined site”.

In order to enable a break at a predetermined target site, the enzymes preferably include a binding/recognition domain and a cleavage domain. Particular enzymes capable of inducing double or single-stranded breaks are nucleases or nickases as well as variants thereof, including such molecules no longer comprising a nuclease or nickase function but rather operating as recognition molecules in combination with another enzyme. In recent years, many suitable nucleases, especially tailored endonucleases have been developed comprising meganucleases, zinc finger nucleases, TALE nucleases, Argonaute nucleases, derived, for example, from Natronobacterium gregoryi, and CRISPR nucleases, comprising, for example, Cas9, Cpf1, CasX or CasY nucleases as part of the Clustered Regularly Interspaced Short Palindromic Repeats (CRISPR) system. Thus, in a preferred aspect of the invention, the genome engineering component comprises a DSB or SSB inducing enzyme or a variant thereof selected from a CRISPR/Cas endonuclease, preferably a CRISPR/Cas9 endonuclease or a CRISPR/Cpf1 endonuclease, a zinc finger nuclease (ZFN), a homing endonuclease, a meganuclease and a TAL effector nuclease.

Rare-cleaving endonucleases are DSBI/SSBI enzymes that have a recognition site of preferably about 14 to 70 consecutive nucleotides, and therefore have a very low frequency of cleaving, even in larger genomes such as most plant genomes. Homing endonucleases, also called meganucleases, constitute a family of such rare-cleaving endonucleases. They may be encoded by introns, independent genes or intervening sequences, and present striking structural and functional properties that distinguish them from the more classical restriction enzymes, usually from bacterial restriction-modification Type II systems. Their recognition sites have a general asymmetry which contrast to the characteristic dyad symmetry of most restriction enzyme recognition sites. Several homing endonucleases encoded by introns or inteins have been shown to promote the homing of their respective genetic elements into allelic intronless or inteinless sites. By making a site-specific double strand break in the intronless or inteinless alleles, these nucleases create recombinogenic ends, which engage in a gene conversion process that duplicates the coding sequence and leads to the insertion of an intron or an intervening sequence at the DNA level. A list of other rare cleaving meganucleases and their respective recognition sites is provided in Table I of WO 03/004659 (pages 17 to 20) (incorporated herein by reference).

Furthermore, methods are available to design custom-tailored rare-cleaving endonucleases that recognize basically any target nucleotide sequence of choice. Briefly, chimeric restriction enzymes can be prepared using hybrids between a zinc-finger domain designed to recognize a specific nucleotide sequence and the non-specific DNA-cleavage domain from a natural restriction enzyme, such as Fokl. Such methods have been described e.g. in WO 03/080809, WO 94/18313 or WO 95/09233 and in Isalan et al. (2001). A rapid, generally applicable method to engineer zinc fingers illustrated by targeting the HIV-1 promoter. Nature biotechnology, 19(7), 656; Liu et al. (1997). Design of polydactyl zinc-finger proteins for unique addressing within complex genomes. Proceedings of the National Academy of Sciences, 94(11), 5525-5530.).

Another example of custom-designed endonucleases includes the TALE nucleases (TALENs), which are based on transcription activator-like effectors (TALEs) from the bacterial genus Xanthomonas fused to the catalytic domain of a nuclease (e.g. Fokl or a variant thereof). The DNA binding specificity of these TALEs is defined by repeat-variable di-residues (RVDs) of tandem-arranged 34/35-amino acid repeat units, such that one RVD specifically recognizes one nucleotide in the target DNA. The repeat units can be assembled to recognize basically any target sequences and fused to a catalytic domain of a nuclease create sequence specific endonucleases (see e.g. Boch et al. (2009). Breaking the code of DNA binding specificity of TAL-type III effectors. Science, 326(5959), 1509-1512; Moscou & Bogdanove (2009). A simple cipher governs DNA recognition by TAL effectors. Science, 326(5959), 1501-1501; and WO 2010/079430, WO 2011/072246, WO 2011/154393, WO 2011/146121, WO 2012/001527, WO 2012/093833, WO 2012/104729, WO 2012/138927, WO 2012/138939). WO 2012/138927 further describes monomeric (compact) TALENs and TALEs with various catalytic domains and combinations thereof.

Recently, a new type of customizable endonuclease system has been described; the so-called CRISPR/Cas system. A CRISPR system in its natural environment describes a molecular complex comprising at least one small and individual non-coding RNA in combination with a Cas nuclease or another CRISPR nuclease like a Cpf1 nuclease (Zetsche et al., “Cpf1 Is a Single RNA-Guides Endonuclease of a Class 2 CRISPR-Cas System”, Cell, 163, pp. 1-13, October 2015) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbiol., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems code for a Cas9 nuclease as key enzyme for the interference step, which system contains both a crRNA and also a trans-activating RNA (tracrRNA) as the guide motif. These hybridize and form double-stranded (ds) RNA regions which are recognized by RNAselll and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease. As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337: 816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

The cleavage site of a DSBI/SSBI enzyme relates to the exact location on the DNA or RNA where the break is induced. The cleavage site may or may not be comprised in (overlap with) the recognition site of the DSBI/SSBI enzyme and hence it is said that the cleavage site of a DSBI/SSBI enzyme is located at or near its recognition site. The recognition site of a DSBI/SSBI enzyme, also sometimes referred to as binding site, is the nucleotide sequence that is (specifically) recognized by the DSBI/SSBI enzyme and determines its binding specificity. For example, a TALEN or ZNF monomer has a recognition site that is determined by their RVD repeats or ZF repeats respectively, whereas its cleavage site is determined by its nuclease domain (e.g. Fokl) and is usually located outside the recognition site. In case of dimeric TALENs or ZFNs, the cleavage site is located between the two recognition/binding sites of the respective monomers, this intervening DNA or RNA region where cleavage occurs being referred to as the spacer region.

A person skilled in the art would be able to either choose a DSBI/SSBI enzyme recognizing a certain recognition site and inducing a DSB or SSB at a cleavage site at or in the vicinity of the preselected/predetermined site or engineer such a DSBI/SSBI enzyme. Alternatively, a DSBI/SSBI enzyme recognition site may be introduced into the target genome using any conventional transformation method or by crossing with an organism having a DSBI/SSBI enzyme recognition site in its genome, and any desired nucleic acid may afterwards be introduced at or in the vicinity of the cleavage site of that DSBI/SSBI enzyme.

There are two major and distinct pathways to repair breaks—homologous recombination and non-homologous end-joining (NHEJ). Homologous recombination requires the presence of a homologous sequence as a template (e.g., “donor”) to guide the cellular repair process and the results of the repair are error-free and predictable. In the absence of a template (or “donor”) sequence for homologous recombination, the cell typically attempts to repair the break via the process of non-homologous end-joining (NHEJ).

In a particularly preferred aspect of this embodiment, a repair nucleic acid molecule is additionally introduced into the plant cell. As used herein, a “repair nucleic acid molecule” is a single-stranded or double-stranded DNA molecule or RNA molecule that is used as a template for modification of the genomic DNA or the RNA at the preselected site in the vicinity of or at the cleavage site. As used herein, “use as a template for modification of the genomic DNA”, means that the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two end of the repair nucleic acid molecule (e.g. in case there is only one flanking region). Integration by homologous recombination will allow precise joining of the repair nucleic acid molecule to the target genome up to the nucleotide level, while NHEJ may result in small insertions/deletions at the junction between the repair nucleic acid molecule and genomic DNA.

As used herein, “a modification of the genome”, means that the genome has changed by at least one nucleotide. This can occur by insertion of a transgene, preferably an expression cassette comprising a transgene of interest, replacement of at least one nucleotide and/or a deletion of at least one nucleotide and/or an insertion of at least one nucleotide, as long as it results in a total change of at least one nucleotide compared to the nucleotide sequence of the preselected genomic target site before modification, thereby allowing the identification of the modification, e.g. by techniques such as sequencing or PCR analysis and the like, of which the skilled person will be well aware.

As used herein “a preselected site”, “a predetermined site” or “predefined site” indicates a particular nucleotide sequence in the genome (e.g. the nuclear genome or the chloroplast genome) at which location it is desired to insert, replace and/or delete one or more nucleotides. This can e.g. be an endogenous locus or a particular nucleotide sequence in or linked to a previously introduced foreign DNA, RNA or transgene. The preselected site can be a particular nucleotide position at (after) which it is intended to make an insertion of one or more nucleotides. The preselected site can also comprise a sequence of one or more nucleotides which are to be exchanged (replaced) or deleted.

As used in the context of the present application, the term “about” means +/−10% of the recited value, preferably +/−5% of the recited value. For example, about 100 nucleotides (nt) shall be understood as a value between 90 and 110 nt, preferably between 95 and 105.

As used herein, a “flanking region”, is a region of the repair nucleic acid molecule having a nucleotide sequence which is homologous to the nucleotide sequence of the DNA region flanking (i.e. upstream or downstream) of the preselected site. It will be clear that the length and percentage sequence identity of the flanking regions should be chosen such as to enable homologous recombination between said flanking regions and their corresponding DNA region upstream or downstream of the preselected site. The DNA region or regions flanking the preselected site having homology to the flanking DNA region or regions of the repair nucleic acid molecule are also referred to as the homology region or regions in the genomic DNA.

To have sufficient homology for recombination, the flanking DNA regions of the repair nucleic acid molecule may vary in length, and should be at least about 10 nt, about 15 nt, about 20 nt, about 25 nt, about 30 nt, about 40 nt or about 50 nt in length. However, the flanking region may be as long as is practically possible (e.g. up to about 100-150 kb such as complete bacterial artificial chromosomes (BACs). Preferably, the flanking region will be about 50 nt to about 2000 nt, e.g. about 100 nt, 200 nt, 500 nt or 1000 nt. Moreover, the regions flanking the DNA of interest need not be identical to the homology regions (the DNA regions flanking the preselected site) and may have between about 80% to about 100% sequence identity, preferably about 95% to about 100% sequence identity with the DNA regions flanking the preselected site. The longer the flanking region, the less stringent the requirement for homology. Furthermore, to achieve exchange of the target DNA sequence at the preselected site without changing the DNA sequence of the adjacent DNA sequences, the flanking DNA sequences should preferably be identical to the upstream and downstream DNA regions flanking the preselected site.

As used herein, “upstream” indicates a location on a nucleic acid molecule which is nearer to the 5′ end of said nucleic acid molecule. Likewise, the term “downstream” refers to a location on a nucleic acid molecule which is nearer to the 3′ end of said nucleic acid molecule. For avoidance of doubt, nucleic acid molecules and their sequences are typically represented in their 5′ to 3′ direction (left to right).

In order to target sequence modification at the preselected site, the flanking regions must be chosen so that 3′ end of the upstream flanking region and/or the 5′ end of the downstream flanking region align(s) with the ends of the predefined site. As such, the 3′ end of the upstream flanking region determines the 5′ end of the predefined site, while the 5′ end of the downstream flanking region determines the 3′ end of the predefined site.

As used herein, said preselected site being located outside or away from said cleavage (and/or recognition) site, means that the site at which it is intended to make the genomic modification (the preselected site) does not comprise the cleavage site and/or recognition site of the DSBI/SSBI enzyme, i.e. the preselected site does not overlap with the cleavage (and/or recognition) site. Outside/away from in this respect thus means upstream or downstream of the cleavage (and/or recognition) site.

A “base editor” as used herein refers to a protein or a fragment thereof having the same catalytical activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Preferably, the at least one base editor according to the present invention is temporarily or permanently linked to at least one site-specific DSBI/SSBI enzyme complex or at least one modified site-specific DSBI/SSBI enzyme complex, or optionally to a component of said at least one site-specific DSBI/SSBI enzyme complex. The linkage can be covalent and/or non-covalent.

Any base editor or site-specific DSBI/SSBI enzyme complex, or a catalytically active fragment thereof, or any component of a base editor complex or of a site-specific DSBI/SSBI enzyme complex as disclosed herein can be introduced into a cell as a nucleic acid fragment, the nucleic acid fragment representing or encoding a DNA, RNA or protein effector, or it can be introduced as DNA, RNA and/or protein, or any combination thereof.

The base editor is a protein or a fragment thereof having the capacity to mediate a targeted base modification, i.e., the conversion of a base of interest resulting in a point mutation of interest. Preferably, the at least one base editor in the context of the present invention is temporarily or permanently fused to at least one DSBI/SSBI enzyme, or optionally to a component of at least one DSBI/SSBI. The fusion can be covalent and/or non-covalent. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalyzed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al ((2017). Programmable base editing of A·T to G·C in genomic DNA without DNA cleavage. Nature, 551(7681), 464.) described adenine base editors (ABEs) that mediate the conversion of A·T to G·C in genomic DNA.

Enzymes effecting DNA methylation, as well as histone-modifying enzymes have been identified in the art. Histone posttranslational modifications play significant roles in regulating chromatin structure and gene expression. For example, enzymes for histone acetylation are described in Sterner D E, Berger S L (June 2000): “Acetylation of histones and transcription-related factors”, Microbiol. Mol. Biol. Rev. 64 (2): 435-59. Enzymes effecting histone methylation are described in Zhang Y, Reinberg D (2001): “Transcription regulation by histone methylation: interplay between different covalent modifications of the core histone tails”, Genes Dev. 15 (18): 2343-60. Histone ubiquitination is described in Shilatifard A (2006): “Chromatin modifications by methylation and ubiquitination: implications in the regulation of gene expression”. Annu. Rev. Biochem. 75: 243-69. Enzymes for histone phosphorylation are described in Nowak S J, Corces V G (April 2004): “Phosphorylation of histone H3: a balancing act between chromosome condensation and transcriptional activation”, Trends Genet. 20 (4): 214-20. Enzymes for histone sumoylation are described in Nathan D, Ingvarsdottir K, Sterner D E, et al. (April 2006): “Histone sumoylation is a negative regulator in Saccharomyces cerevisiae and shows dynamic interplay with positive-acting histone modifications”, Genes Dev. 20 (8): 966-76. Enzymes for histone ribosylation are described in Hassa P O, Haenni S S, Elser M, Hottiger M O (September 2006): “Nuclear ADP-ribosylation reactions in mammalian cells: where are we today and where are we going?”, Microbiol. Mol. Biol. Rev. 70 (3): 789-829. Histone citrullination is catalyzed for example by an enzyme called peptidylarginine deiminase 4 (PAD4, also called PADI4), which converts both histone arginine (Arg) and mono-methyl arginine residues to citrulline.

Enzymes effecting DNA methylation and histone-modifying enzymes may be fused to a disarmed DSB or SSB inducing enzyme, which preferably recognizes a predetermined site in the genome of said cell.

Epigenetically Regulating Chemicals

As used herein, “epigenetically regulating chemicals” refers to any chemicals involved in regulating the epigenetic status of plant cells, e.g. DNA methylation, protein methylation, in particular histone methylation, and acetylation, in particular histone acetylation. According to a first embodiment of the present invention, a epigenetically regulating chemical, e.g. protein deacetylase inhibitor (ii.1) is co-introduced with the genome engineering component. Preferred epigenetically regulating chemicals for use according to the invention are histone deacetylase inhibitors (HDACIs) such as trichostatin A (TSA) or DNA methyltransferase inhibitor. As used herein, “Histone deacetylase inhibitor (HDACI)” refers to any materials that repress histone deacetylase activity, “DNA methyltransferase inhibitor” refers to any materials that repress DNA methyltransferase activity.

It is assumed that the co-delivered epigenetically regulating chemicals (ii.1) (in particular HADCis) relax plant chromatin structure, promote the DNA accessibility to the genome engineering components in the bombarded cells, thus consequently promote genome engineering (i.e. transformation and genome editing) efficiencies. The reason for this assumption is: The basic structural and functional unit of genetic material is the nucleosome, in which negatively charged DNA is wrapped around a positively charged histone octamer and associated linker histones. Nucleosome units further fold and pack into chromatin (Andrews, A. J., and Luger, K. (2011). Nucleosome structure(s) and stability: Variations on a theme. Annu. Rev. Biophys. 40: 99-117.). DNA accessibility largely depends on compactness of the nucleosomes and chromatins. Chromatin-remodeling enzymes dynamically modify lysine or other amino acids of histones, which cause changes in their charges and interactions with DNA and other proteins, and result in chromatin folding or unfolding (Bannister A J, Kouzarides T (2011) Regulation of chromatin by histone modifications. Cell Res 21: 381-95.). By adding or removing an acetyl group, acetylation and deacetylation of the lysine residue in histone proteins are often involved in the reversible modulation of chromatin structure in eukaryotes, and mediate chromatin accessibility and the regulation of gene expression. Histone deacetylases (HDAC) are enzymes that remove acetyl groups from lysine resides on the N-terminal tail of histones, which makes the histone more positively charged, and therefore allows the histone wrap DNA more tightly. Inhibition of HDACs might help chromatin unfolding and enable the DNA to be more accessible.

Chromatin remodeling and other epigenetic modifications surely play an important role in regulating cell totipotency and regeneration (Zhang, H., and Ogas, J. (2009). An epigenetic perspective on developmental regulation of seed genes. Mol. Plant 2: 610-627.). Inhibition of histone deacetylase (HDAC) activities have been shown associated with plant regeneration and microspore embryogenesis (Miguel, C., and L. Marum. 2011. An epigenetic view of plant cells cultured in vitro: somaclonal variation and beyond. J. Exp. Bot. 62:3713-3725., Li Hui et al. (2014) The Histone Deacetylase Inhibitor Trichostatin A Promotes Totipotency in the Male Gametophyte PLANT CELL, 26: 195-209.). Inhibition of HDAC activity or downstream HDAC-mediated pathways plays a major role in the initiation of stress-induced haploid embryogenesis. One such HDACi is trichostatin A (TSA). It has been shown that TSA induces massive embryogenic cell proliferation in the male gametophyte of B. napus. TSA treatment leads to a high frequency of sporophytic cell division in cultured microspores and pollen.

This document describes methods to increase genome engineering efficiency in presence of one or more epigenetically regulating chemicals, e.g. protein deacetylase inhibitors, in particular HDACi. Such an HDACi may be trichostatin A (TSA), N-Hydroxy-7-(4-dimethylaminobenzoyl)-aminoheptanamide (M344), suberoylanilide hydroxamic acid (SAHA), or others. These HDACIs are selected from hydroxamic acid (HA)-based chemicals, which target to zinc dependent HDACs.

Phytohormones

According to a second embodiment of the invention, one or more phytohormones (ii.2), such as auxins and cytokinins like 2,4-D, 6-Benzylaminopurine (6-BA) and Zeatin, are co-delivered with the genome engineering component (i). As used herein, “phytohormones” refers to any materials and chemicals, either naturally occurred or synthesized, which promote plant cell division and/or plant morphogenesis.

Plant somatic cells are capable to resume cell division and regenerate into an entire plant in in-vitro culture through somatic embryogenesis or organogenesis, which largely depends on phytohormones, such as auxins and cytokinins. In the present invention it was found, that phytohormones promote cell proliferation, increase the sensitivity of the plant cells to genome engineering, and thus improve genome engineering (i.e. transformation and genome editing) efficiency.

One of auxins is 2,4-Dichlorophenoxyacetic acid (2,4-D), which is nearly indispensable for somatic embryogenesis and cell regeneration in monocot plants, e.g. maize and wheat. Meanwhile, cytokinins e.g. 6 benzylaminopurine (6-BA) or Zeatin, are essential for plant organogenesis, and shoot meristem initiation and development. This document describes methods to improve genome engineering efficiency by co-delivery of one or more of phytohormones (2,4-D, 6-BA, Zeatin, etc.) with a genome engineering component.

Regeneration Boost Genes

According to a third embodiment of the invention, a protein causing improved plant regeneration from a somatic cell, a callus cell or an embryonic cell or an expression cassette comprising a nucleic acid encoding the protein (ii.3) is co-introduced with the genome engineering component (i). This type of compounds (ii.3) is also called herein “regeneration boost gene”.

It is believed that transformed cells are less regenerable than wild type cells. Transformed cells are susceptible to programmed cell death due to presence of foreign DNA inside of the cells. Stresses arisen from delivery (e.g. bombardment damage) may trigger a cell death as well. Therefore, promoting cell division is essential for the regeneration of the modified cells. Further, genome engineering efficiency is controlled largely by host cell statuses. The cells undergoing rapid cell-division, like those in plant meristem, are the most suitable recipients for genome engineering. Promoting cell division will probably increase DNA integration or modification during DNA replication and division process, and thus increase genome engineering efficiency.

Boost genes are selected based on their functions involved in promoting cell division and plant morphogenesis. Each of the candidate genes are cloned and driven by a strong constitutive promoter, and evaluated by transient expression in corn cells without a selection. Examples for boost genes are PLT5 (PLETHORAS; SEQ ID NOs: 1, 2, 13 and 14), PLT7 (PLETHORA7; SEQ ID NOs: 3, 4, 15 and 16) and RKD genes (RKD2: SEQ ID NOs: 5, 6, 29, 30, 31 and 32; RKD4: SEQ ID Nos: 11, 12, 17, 18, 27 and 28; e.g., Waki, T., Hiki, T., Watanabe, R., Hashimoto, T., & Nakajima, K. (2011). The Arabidopsis RWP-RK protein RKD4 triggers gene expression and pattern formation in early embryogenesis. Current Biology, 21(15), 1277-1281).

PLT (PLETHORA), also called AIL (AINTEGUMENT-LIKE) genes, are members of the AP2 family of transcriptional regulators. Members of the AP2 family of transcription factors play important roles in cell proliferation and embryogenesis in plants (El Ouakfaoui, S., Schnell, J., Abdeen, A., Colville, A., Labbé, H., Han, S., Baum, B., Laberge, S., Miki, B (2010) Control of somatic embryogenesis and embryo development by AP2 transcription factors. PLANT MOLECULAR BIOLOGY 74(4-5):313-326.). PLT genes are expressed mainly in developing tissues of shoots and roots, and required for stem cell homeostasis, cell division and regeneration, and for patterning of organ primordia.

PLT family comprises an AP2 subclade of six members. Four PLT members, PLT1/AIL3 (SEQ ID NOs: 19 and 20), PLT2/AIL4 (SEQ ID NOs: 21 and 22), PLT3/AIL6 (SEQ ID NOs: 9, 10, 23 and 24), and BBM/PLT4/AIL2 (SEQ ID NOs: 7, 8, 25 and 26), are expressed partly overlap in root apical meristem (RAM) and required for the expression of QC (quiescent center) markers at the correct position within the stem cell niche. These genes function redundantly to maintain cell division and prevent cell differentiation in root apical meristem.

Three PLT genes, PLT3/AIL6, PLT5/AIL5, and PLT7/AIL7, are expressed in shoot apical meristem (SAM), where they function redundantly in the positioning and outgrowth of lateral organs. PLT3, PLT5, and PLT7, regulate de novo shoot regeneration in Arabidopsis by controlling two distinct developmental events. PLT3, PLT5, and PLT7 required to maintain high levels of PIN1 expression at the periphery of the meristem and modulate local auxin production in the central region of the SAM which underlies phyllotactic transitions. Cumulative loss of function of these three genes causes the intermediate cell mass, callus, to be incompetent to form shoot progenitors, whereas induction of PLT5 or PLT7 can render shoot regeneration in a hormone-independent manner. PLT3, PLT5, PLT7 regulate and require the shoot-promoting factor CUP-SHAPED COTYLEDON2 (CUC2) to complete the shoot-formation program. PLT3, PLT5, and PLT7, are also expressed in lateral root founder cells, where they redundantly activate the expression of PLT1 and PLT2, and consequently regulate lateral root formation.

According to the present invention, a protein causes improved plant regeneration from a somatic cell, a callus cell or an embryonic cell, preferably comprises an amino acid sequence which is selected from
a) a sequence as set forth in any of SEQ ID NO: 1, 3, 5, 7, 9, 11, 13, 15, 17, 19, 21, 23, 25, 27, 29 or 31,
b) a sequence having an identity of at least 60% to the sequence of (a),
c) a sequence encoded by a nucleic acid sequence as set forth in any of SEQ ID NO: 2, 4, 6, 8, 10, 12, 14, 16, 18, 20, 22, 24, 26, 28, 30 or 32, and
d) a sequence encoded by a nucleic acid sequence having an identity of at least 60% to the nucleic acid sequence of (c),
e) a sequence encoded by a nucleic acid sequence hybridizing under stringent condition with a sequence complementary to the nucleic acid sequence as defined in c).

For the above amino acid sequence of (b) or the nucleic acid sequence of (d), sequence identity is preferably at least 70%, at least 75%, at least 80%, more preferably at least 85%, at least 90%, at least 95%, at least 98% or at least 99% over the whole length of the sequence.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as program NEEDLE as implemented in the The European Molecular Biology Open Software Suite (EMBOSS), e.g. version 6.3.1.2 (Trends in Genetics 16 (6), 276 (2000)), with its default parameter, e.g. for proteins matrix=EBLOSUM62, gapopen=10.0 and gapextend=0.5.

As used herein, the term “hybridize(s)(ing)” refers to the formation of a hybrid between two nucleic acid molecules via base-pairing of complementary nucleotides. The term “hybridize(s)(ing) under stringent conditions” means hybridization under specific conditions. An example of such conditions includes conditions under which a substantially complementary strand, namely a strand composed of a nucleotide sequence having at least 80% complementarity, hybridizes to a given strand, while a less complementary strand does not hybridize. Alternatively, such conditions refer to specific hybridizing conditions of sodium salt concentration, temperature and washing conditions. As an example, highly stringent conditions comprise incubation at 42° C., 50% formamide, 5×SSC (150 mM NaCl, 15 mM trisodium citrate), 50 mM sodium phosphate, 5×Denhardt's solution, 10×dextran sulphate, 20 mg/ml sheared salmon sperm DNA and washing in 0.2×SSC at about 65° C. (SSC stands for 0.15 M sodium chloride and 0.015 M trisodium citrate buffer). Alternatively, highly stringent conditions may mean hybridization at 68° C. in 0.25 M sodium phosphate, pH 7.2, 7% SDDS, 1 mM EDTA and 1% BSA for 16 hours and washing twice with 2×SSC and 0.1% SDDs at 68° C. Further alternatively, highly stringent hybridisation conditions are, for example: Hybridizing in 4×SSC at 65° C. and then multiple washing in 0.1×SSC at 65° C. for a total of approximately 1 hour, or hybridizing at 68° C. in 0.25 M sodium phosphate, pH 7.2, 7% SDS, 1 mM EDTA and 1% BSA for 16 hours and subsequent washing twice with 2×SSC and 0.1% SDS at 68° C.

Co-Introduction

The method of the invention for genetic modification in a plant cell is characterized in that a genome engineering component (i) and at least one of compounds (ii.1), (ii.2) and (ii.3) are co-introduced into one plant cell.

As used herein, “co-delivery” or “co-deliver” and “co-introduction” or “co-introduce” are used interchangeably. In terms of the present invention, “co-introducing” refers to the process, in which at least two different components are delivered into the same plant cell concurrently. Thus, the genome engineering component (i) and compounds (ii.1), (ii.2) and/or (ii.3) are introduced together into the same plant cell. Preferably, both types of components/compounds are introduced via a common construct.

Co-introduction into the plant cell can be conducted by particle bombardment, microinjection, agrobacterium-mediated transformation, electroporation, agroinfiltration or vacuum infiltration. According to the invention, methods based on physical delivery like particle bombardment, microinjection, electroporation, nanoparticles, and cell-penetrating peptides (CPPs) are particularly preferred for co-introducing components (i) and compounds (ii). Particularly preferred is the co-introduction via particle bombardment.

The term “particle bombardment” as used herein, also named “biolistic transfection” or “microparticle-mediated gene transfer” refers to a physical delivery method for transferring a coated microparticle or nanoparticle comprising a construct of interest into a target cell or tissue. For use in the present invention, the construct of interest comprises the genome engineering component (i) and at least one of compounds (ii.1), (ii.2), and (ii.3). The micro- or nanoparticle functions as projectile and is fired on the target structure of interest under high pressure using a suitable device, often called gene-gun. The transformation via particle bombardment uses a microprojectile of metal covered with the construct of interest, which is then shot onto the target cells using an equipment known as “gene gun” (Sandford et al. 1987) at high velocity fast enough (1500 km/h) to penetrate the cell wall of a target tissue, but not harsh enough to cause cell death. For protoplasts, which have their cell wall entirely removed, the conditions are different logically. The precipitated construct on the at least one microprojectile is released into the cell after bombardment. The acceleration of microprojectiles is accomplished by a high voltage electrical discharge or compressed gas (helium). Concerning the metal particles used it is mandatory that they are non-toxic, non-reactive, and that they have a lower diameter than the target cell. The most commonly used are gold or tungsten. There is plenty of information publicly available from the manufacturers and providers of gene-guns and associated system concerning their general use.

In a particularly preferred embodiment of microparticle bombardment, one or more compounds (ii.1), (ii.2) and (ii.3) can be co-delivered with the genome engineering component (i) via microcarriers comprising gold particles having a size in a range of 0.4-1.6 micron (μm), preferably 0.4-1.0 μm. In an exemplary process, 10-1000 μg of gold particles, preferably 50-300 μg, are used per one bombardment.

The compounds (ii) and genome engineering component (i) can be delivered into target cells for example using a Bio-Rad PDS-1000/He particle gun or handheld Helios gene gun system. When a PDS-1000/He particle gun system used, the bombardment rupture pressures are from 450 psi to 2200 psi, preferred from 450-1100 psi, while the rupture pressures are from 100-600 psi for a Helios gene gun system. More than one chemical or construct can be co-delivered with genome engineering components into target cells simultaneously.

Cultivation Step

In step b) of the method of the invention, the plant cell into which the genome engineering component (i) and at least one compound (ii) have been co-introduced is cultivated under conditions allowing the genetic modification of the genome of said plant cell by activity of the genome engineering component in the presence of the at least one compound (ii).

As used herein, “genetic modification of the genome” includes any type of manipulation such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetic modification is an alteration in the regulatory sequence, such as a promoter, to result in increased or decreased expression of an operably linked endogenous coding region.

Conditions that are “suitable” for a genetic modification of the plant genome to occur, such as cleavage of a polynucleotide, or “suitable” conditions are conditions that do not prevent such events from occurring. Thus, these conditions permit, enhance, facilitate, and/or are conducive to the event. Depending on the respective genome engineering component (i), these conditions may differ.

In the method of the present invention, the plant cell is preferably transiently transformed with the genome engineering component (i) and the at least one compound (ii). As used herein, “transient transformation” refers to the transfer of a foreign material [i.e. a nucleic acid fragment, protein, ribonucleoprotein (RNP), etc.] into host cells resulting in gene expression and/or activity without integration and stable inheritance of the foreign material. Thus, the genome engineering component (i) is transiently active and/or transiently present in the plant cell. The genome engineering component is not permanently incorporated into the cellular genome, but provides a temporal action resulting in a modification of the genome. For example, transient activity and/or transient presence of the genome engineering component in the plant cell can result in introducing one or more double-stranded breaks in the genome of the plant cell, one or more single-stranded breaks in the genome of the plant cell, one or more base-editing events in the genome of the plant cell, or one or more of DNA methylation, histone acetylation, histone methylation, histone ubiquitination, histone phosphorylation, histone sumoylation, histone ribosylation or histone citrullination in the genome of the plant cell.

The introduction of one or more double-stranded breaks or one or more single-stranded breaks is preferably followed by non-homologous N joining (NHIJ) and/or by homology directed repair of the break(s) through a homologous recombination mechanism.

The resulting modification in the genome of the plant cell can, for example, be selected from an insertion of a transgene, preferably an expression cassette comprising a transgene of interest, a replacement of at least one nucleotide, a deletion of at least one nucleotide, an insertion of at least one nucleotide, a change of DNA methylation, a change in histone acetylation, histone methylation, histone ubiquitination, histone phosphorylation, histone sumoylation, histone ribosylation, or histone citrullination or any combination thereof. According to a particularly preferred aspect of the invention, no exogenous genetic material related to the applied gene editing machinery/systems is stably integrated into the genome of the plant cell.

The genetic modification can be a permanent and heritable change in the genome of the plant cell.

Optional Pre-Treatment

According to a preferred aspect of the invention, a pre-treatment of plant materials with one or more chemicals, e.g. one or more of compounds (ii.1), (ii.2) and (ii.3) can be included before the co-introduction step (a) via in vitro culture of the plant materials in a medium containing the one or more compounds (ii). Thus, the method for genetic modification in a plant cell may further comprise a step of pretreatment of the plant cell to be used in step a), said pretreatment comprising culturing the plant cell or plant material comprising same in a medium containing (ii.1) the epigenetically regulating chemical or an active derivative thereof, in particular the histone deacetylase inhibitor (HDACi) or or the DNA methyltransferase inhibitor, (ii.2) the phytohormone or the active derivative thereof, (ii.3) the protein causing improved plant regeneration from callus tissue or embryonic tissue, or any combination thereof.

After the pretreatment step, the treated plant cells are taken from the medium containing at least one of compounds (ii.1), (ii.2) and (ii.3) and used for co-introduction step (a).

Exemplary, as for the histone deacetylase inhibitor TSA, the duration of the HDACis pre-treatment is from 10 minutes to 2 days, preferred 2.0 to 24 hours. TSA concentration for a pre-treatment is 1.0 nM to 1000 nM, preferred 10 nM to 100 nM. Hereafter the treated plant materials are transferred to HDACi-free medium and used for TSA co-introduction immediately (a prolonged TSA pre-treatment may cause non-selectively enhancement of cell regeneration, which may increase difficult in retrieving the bombarded and modified cells).

Similar conditions of pre-treatment can be applied for all types of compounds (ii.1), (ii.2) and (ii.3). Plant tissue culture and genome engineering can be carried out using currently available methods. Transient transformation and transgene expression may be monitored by use of the red fluorescent report gene tdTomato, which encodes an exceptionally bright red fluorescent protein with excitation maximum at 554 nm and emission maximum at 581 nm, or the green fluorescent report gene mNeonGreen, which encodes the brightest monomeric green or yellow fluorescent protein with excitation maximum at 506 nm and emission maximum at 517 nm. The genome editing efficiency can be analyzed for instance by next generation sequencing (NGS).

Microparticles

In the context of the present invention, it was found that for co-introducing components (i) and (ii) into a plant cell, microparticles which are coated with both components are particularly suitable. Thus, according to another embodiment, the present invention provides a microparticle coated with at least
(i) a genome engineering component and
(ii) a second compound comprising
   (ii.1) an epigenetically regulating chemicals, e.g. protein deacetylase inhibitor or an active derivative thereof, in particular a histone deacetylase inhibitor (HDACi), and/or
   (ii.2) a phytohormone or an active derivative thereof, and/or
   (ii.3) a protein causing improved plant regeneration from a somatic cell, a callus cell or an embryonic cell or an expression cassette comprising a nucleic acid encoding the protein.

The microparticle consists of a non-toxic, non-reactive material. Preferably, the microparticle comprises a metal such as gold or tungsten. The size of the microparticle may be in a range of 0.4-1.6 micron (μm), preferably 0.4-1.0 μm.

The coating with components (i) and (ii) can comprise one or more coating layers. For example, a microparticle may contain a first coating layer comprising genome engineering component (i) and a second coating layer comprising compound (ii.1), (ii.2) and/or (ii.3). Alternatively, a microparticle may contain a coating layer comprising genome engineering component (i) and at least one of compounds (ii.1), (ii.2) and (ii.3).

Further, the invention provides a kit for the genetic modification of a plant genome by microprojectile bombardment, comprising
(I) one or more microparticles, and
(II) means for coating the microparticles with at least a genome engineering component and (1) an epigenetically regulating chemical, e.g. a DNA methyltransferase inhibitor or a protein deacetylase inhibitor or an active derivative thereof, in particular a histone deacetylase inhibitor (HDACi), and/or (2) a phytohormone or an active derivative thereof, and/or (3) a protein causing improved plant regeneration from callus tissue or embryonic tissue or an expression cassette comprising a nucleic acid encoding the protein.

Another aspect of the present invention is the use of a microparticle as described above for the biolistic transformation of a plant cell.

Subject matter of the present invention are also the plant cells that are obtained or obtainable by the methods described above. Accordingly, one embodiment of the invention is a genetically modified plant cell obtained or obtainable by the above method for genetic modification in a plant cell. The genetic modification in these plant cells compared to the original plant cells may, for example, include an insertion of a transgene, preferably an expression cassette comprising a transgene of interest, a replacement of at least one nucleotide, a deletion of at least one nucleotide, an insertion of at least one nucleotide, a change of DNA methylation, a change in histone acetylation, histone methylation, histone ubiquitination, histone phosphorylation, histone sumoylation, histone ribosylation, or histone citrullination or any combination thereof. Preferably, the genetically modified plant cell does not comprise any exogenous genetic materials stably integrated into the genome of the plant cell.

Genetically modified plant cells can be part of a whole plant or part thereof. Thus, the present invention also relates to a plant or plant part comprising the above genetically modified plant cell.

According to another aspect of the present invention, the genetically modified plant cells can be regenerated into a whole (fertile) plant. Thus, in a preferred aspect of the invention, the genetic modification of a plant cell is followed by a step of regenerating a plant. Accordingly, the present invention provides a method for producing a genetically modified plant comprising the steps:
a) genetically modifying a plant cell according to the above method for genetic modification in a plant cell, and
b) regenerating a plant from the modified plant cell of step a),

preferably wherein the produced plant does not contain any of the genome engineering component, the epigenetically regulating chemical or an active derivative thereof, in particular a DNA methyltransferase inhibitor or a histone deacetylase inhibitor (HDACi), the phytohormone or an active derivative thereof, or the protein causing improved plant regeneration from callus tissue or embryonic tissue or the expression cassette comprising a nucleic acid encoding the protein, co-introduced in step a).

As used herein, “regeneration” refers to a process, in which single or multiple cells proliferate and develop into tissues, organs, and eventually entire plants.

Step b) of regenerating a plant can for example comprise culturing the genetically modified plant cell from step a) on a regeneration medium.

Regeneration techniques rely on manipulation of certain phytohormones in a tissue culture growth medium, occasionally relying on a biocide and/or herbicide marker that can been introduced. Regeneration can be obtained from plant somatic cells, callus cells or embryonic cells and protoplasts derived from different explants, e.g. callus, immature or mature embryos, leaves, shoot, roots, flowers, microspores, embryonic tissue, meristematic tissues, organs, or any parts thereof. Such regeneration techniques are described generally in Klee (1987) Ann. Rev. of Plant Phys. 38:467486. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. To obtain whole plants from transformed or gene edited cells, the cells can be grown under controlled environmental conditions in a series of media containing nutrients and hormones, a process known as tissue culture. Once whole plants are generated and produce seed, evaluation of the progeny begins.

The present invention also provides a genetically modified plant obtained or obtainable by the above method for producing a genetically modified plant or a progeny plant thereof.

Further subject matter of the present invention is a plant cell or a seed derived from the above genetically modified plant. Such a plant cell or seed does not contain any of the genome engineering component, the epigenetically regulating chemical or an active derivative thereof, in particular a histone deacetylase inhibitor (HDACi), the phytohormone or an active derivative thereof, and the protein causing improved plant regeneration from callus tissue or embryonic tissue or the expression cassette comprising a nucleic acid encoding the protein.

Further subject matter of the present invention is a plant, plant cell or a seed derived from the above genetically modified cell without a marker gene-based selection. As used herein, “marker gene-based selection” refers to any processes to select, identify and/or purify the modified cells, in particular the transformed, gene edited or base edited cells, from wild-type cells by using an integrated selection marker (gene), e.g. antibiotic resistance gene (e.g. kanamycin resistance gene, hygromycin resistance gene), or herbicide resistance gene (e.g. phosphinothricin resistance gene, glyphosate resistance gene). Without such selection, such a plant, plant cell or seed may not have any of the genome engineering components integrated, and thus may leads to transgene-free genetic modified plants or modified which have integrated solely the transgene of interest.

A further aspect of the present invention is the use of a epigenetically regulating chemical, e.g. a protein deacetylase inhibitor or an active derivative thereof, in particular a histone deacetylase inhibitor (HDACi), and/or a phytohormone or an active derivative thereof, and/or a protein causing improved plant regeneration from a somatic cell, a callus cell or an embryonic cell or an expression cassette comprising a nucleic acid encoding the protein for increasing the efficiency of genetic modification in a plant cell, preferably in the method described hereinabove.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by RDD. Cray, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

All patents, patent applications, and publications or public disclosures (including publications on internet) referred to or cited herein are incorporated by reference in their entirety.

The present invention is further illustrated by the following figures and examples. However, it is to be understood that the invention is not limited to such Examples.

### FIGURES

FIG. 1: pLH-Pat5077399-70Subi-tDt construct map. tDT defines tdTomato gene.

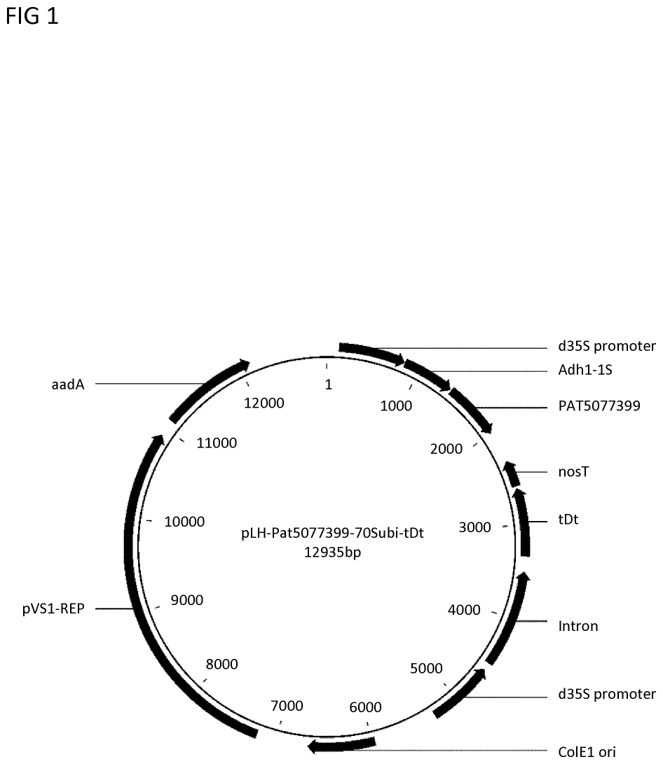

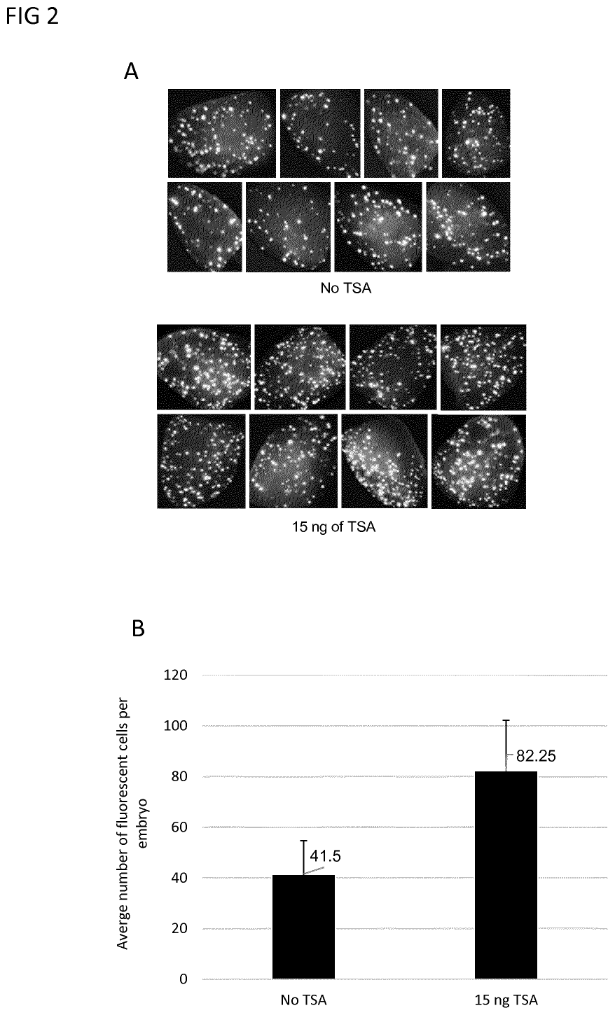

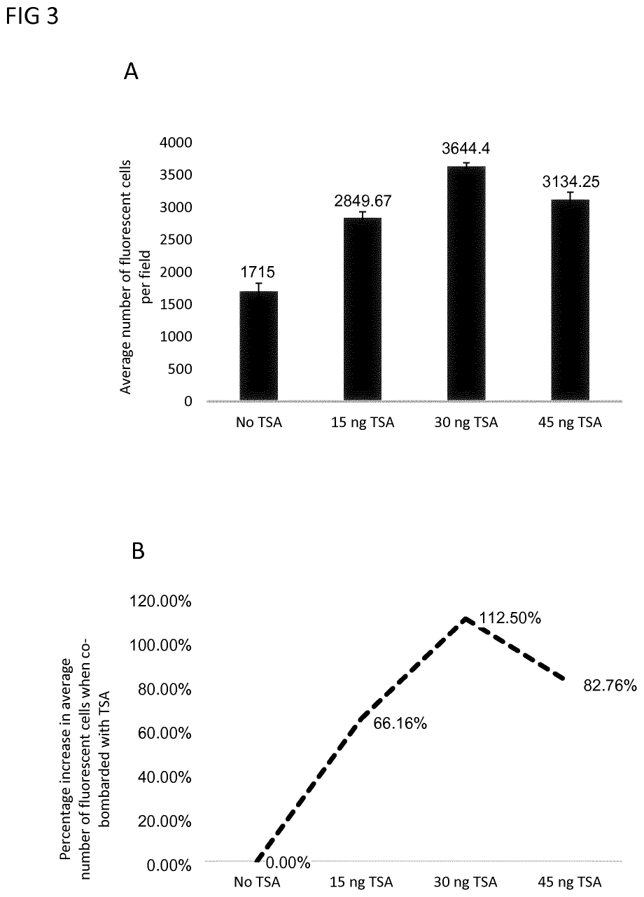

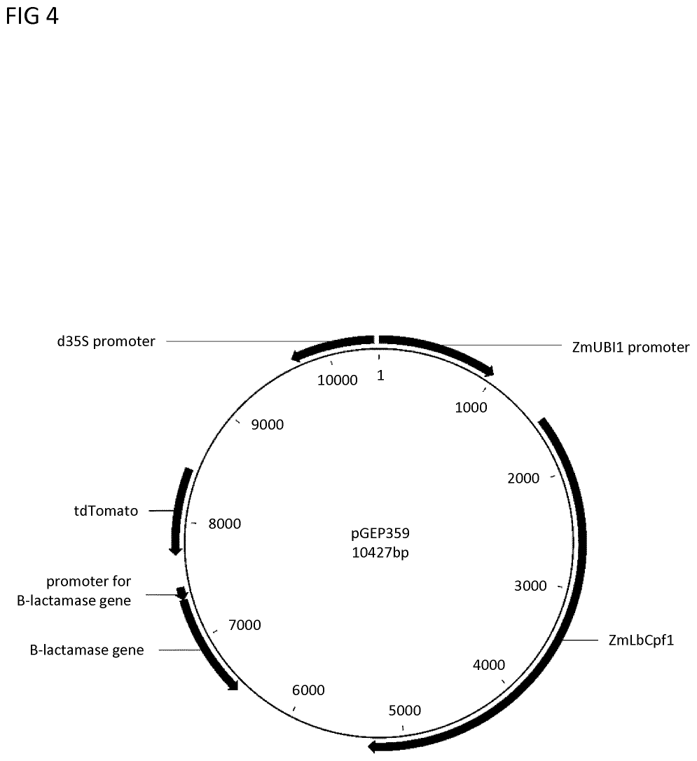

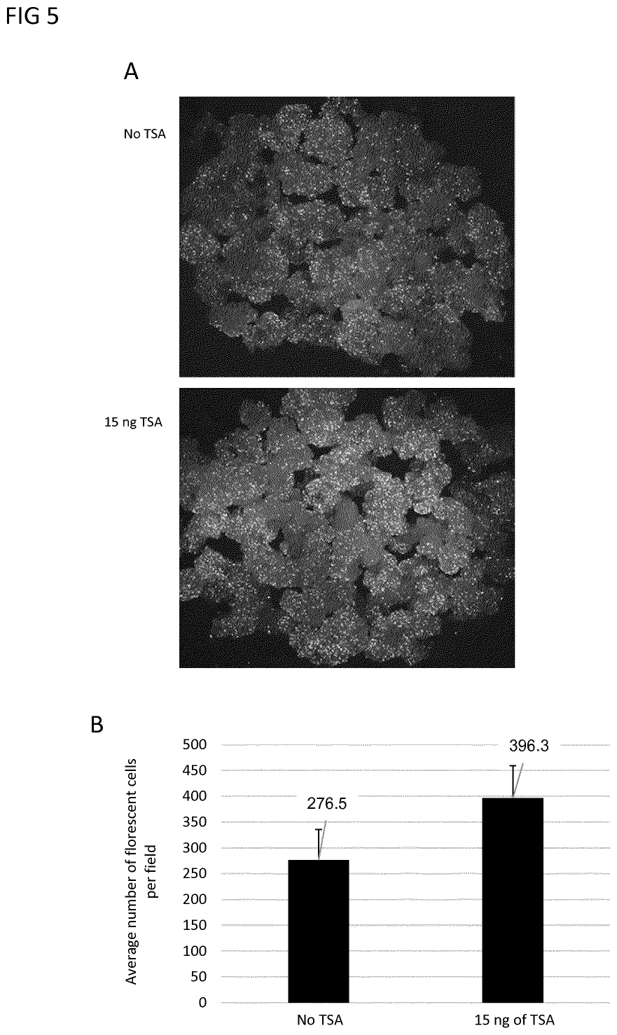

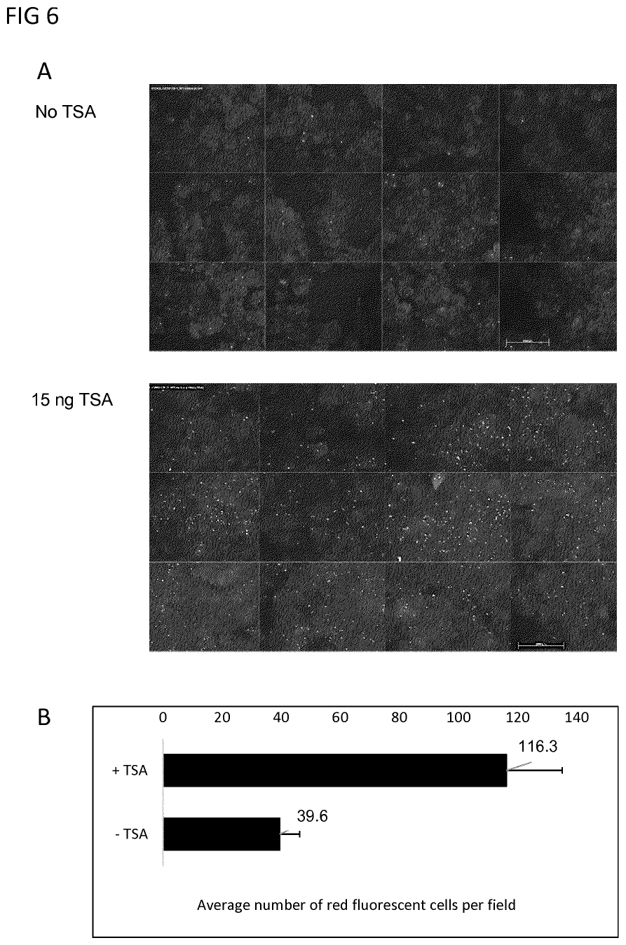

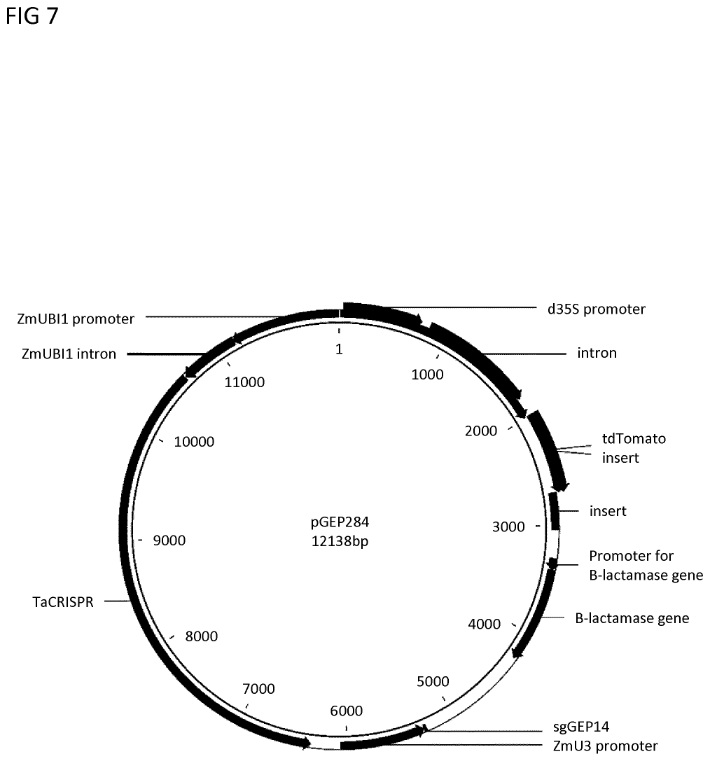

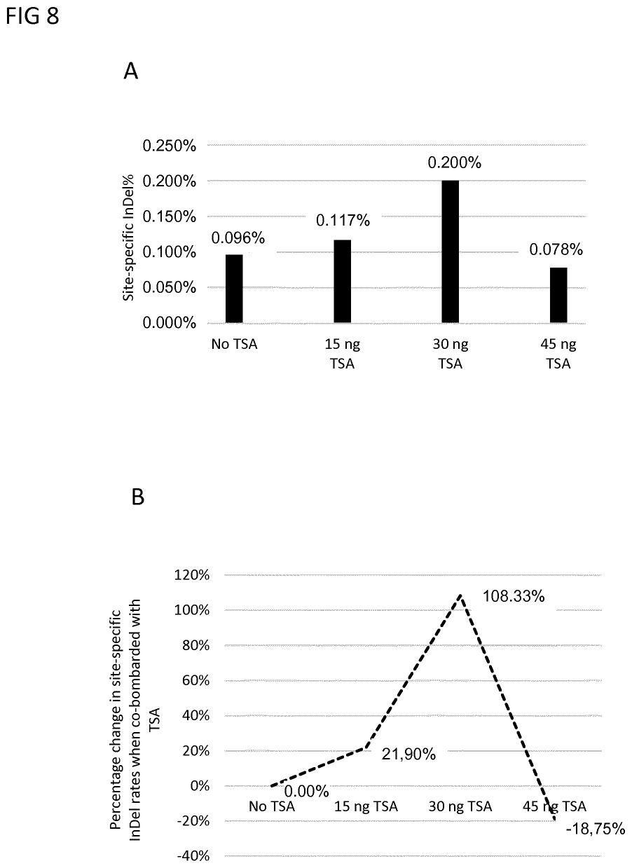

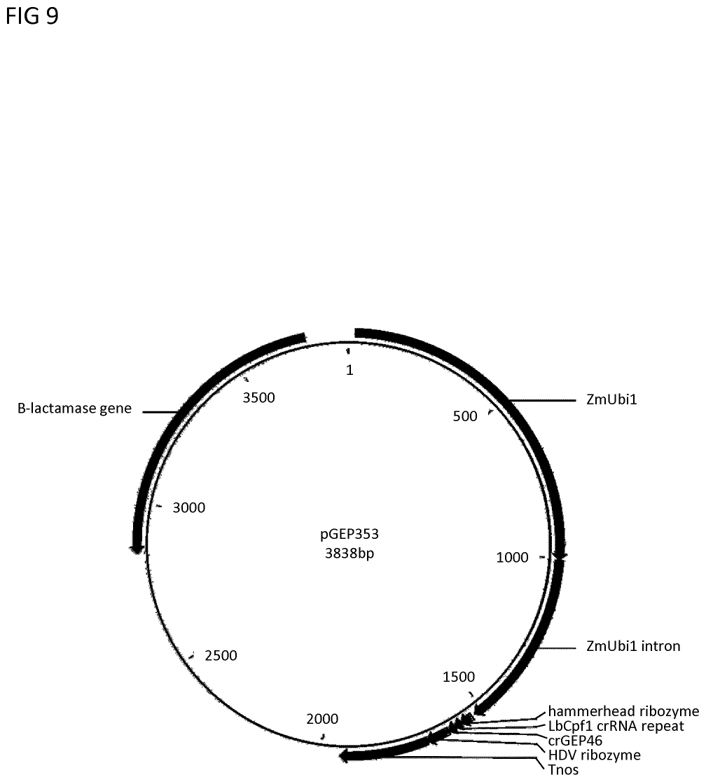

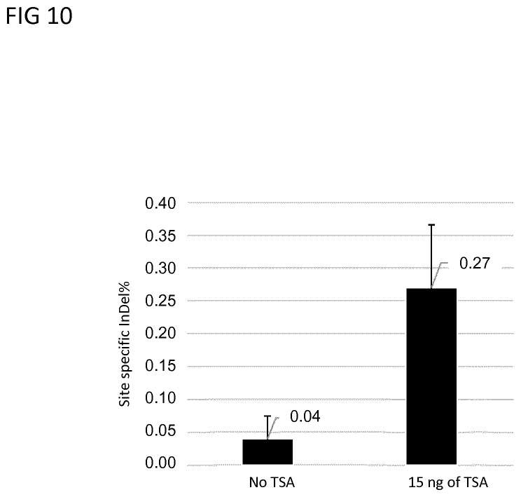

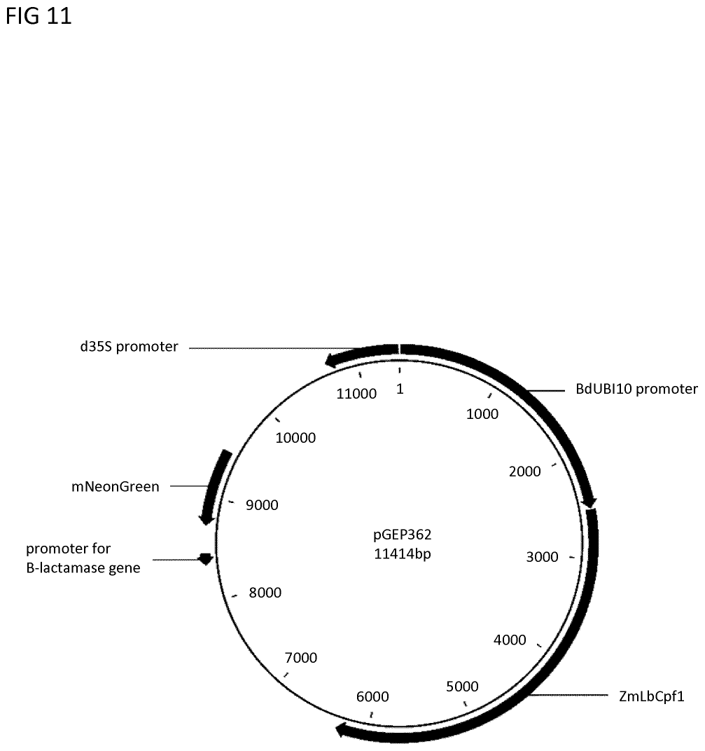

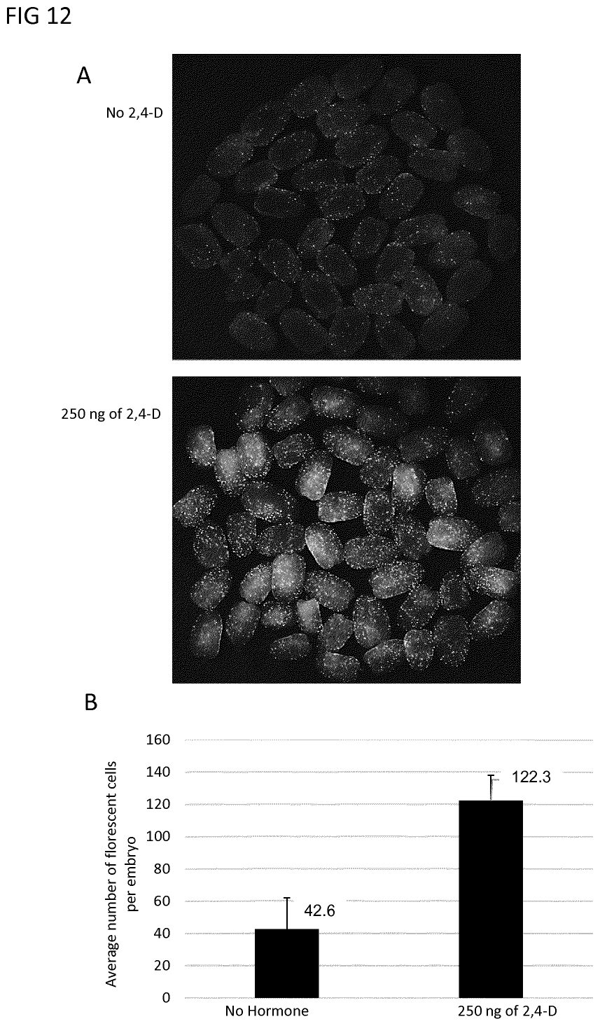

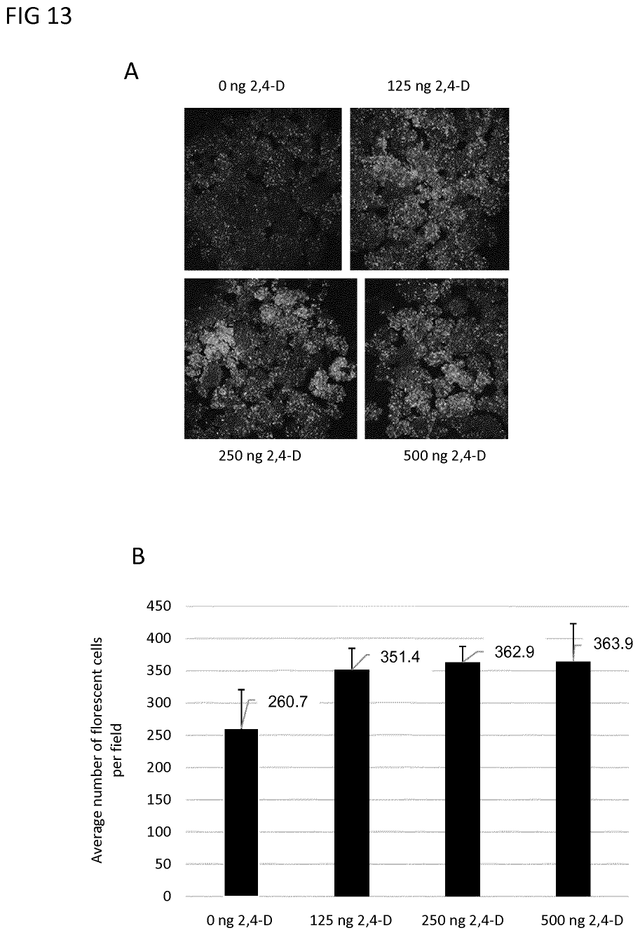

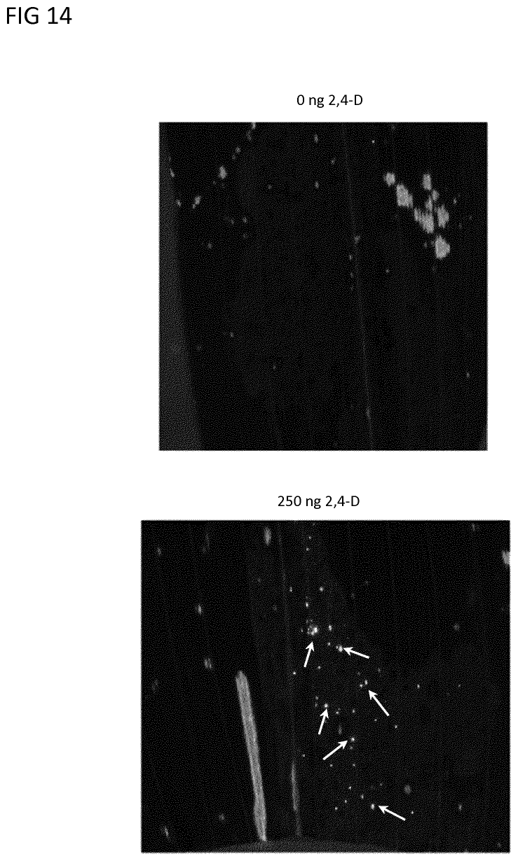

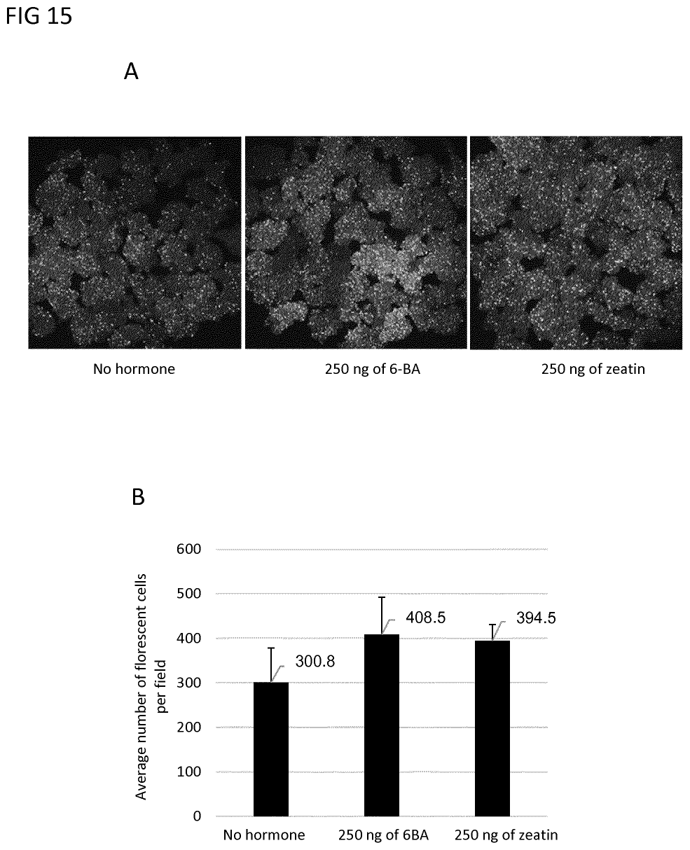

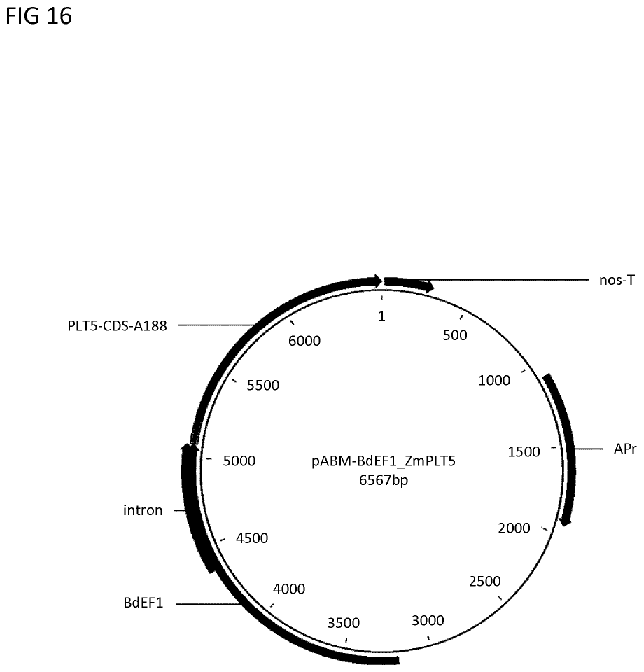

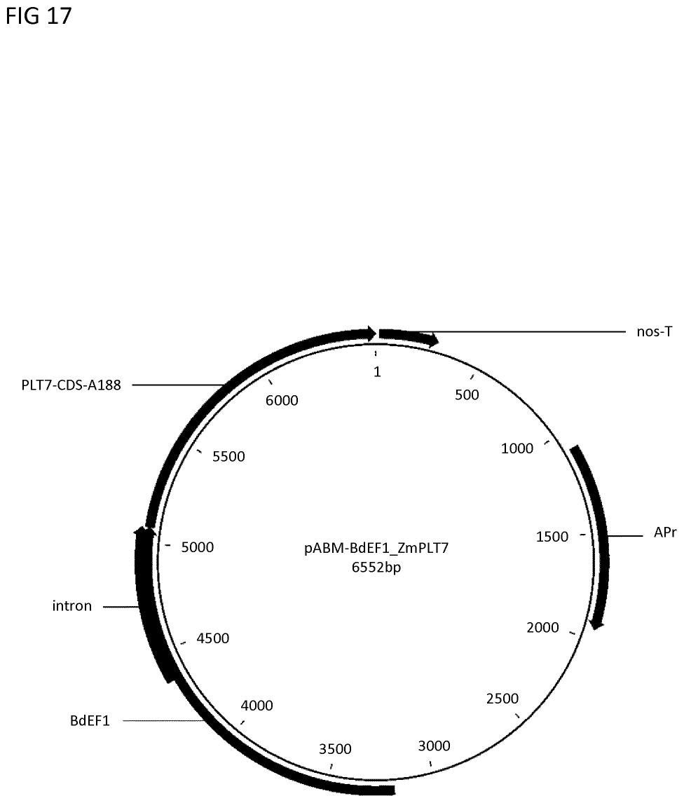

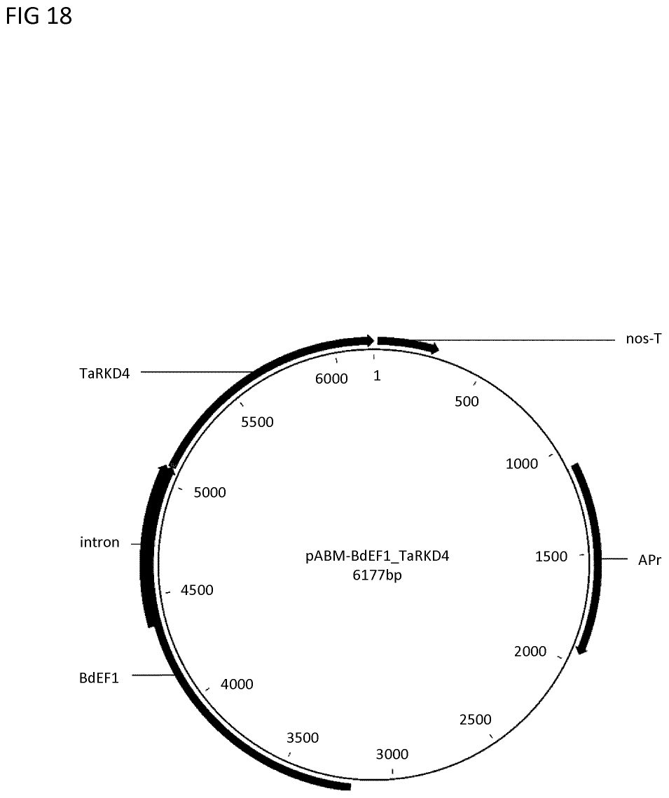

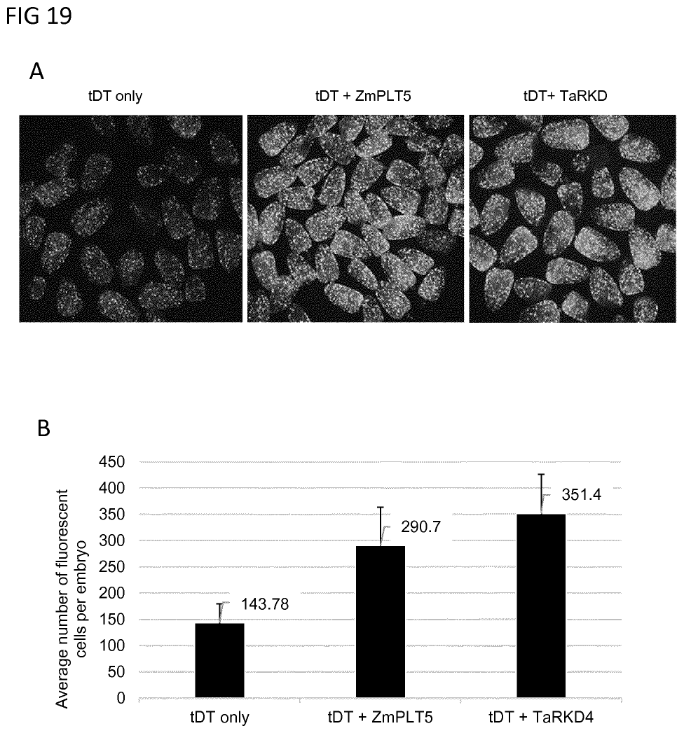

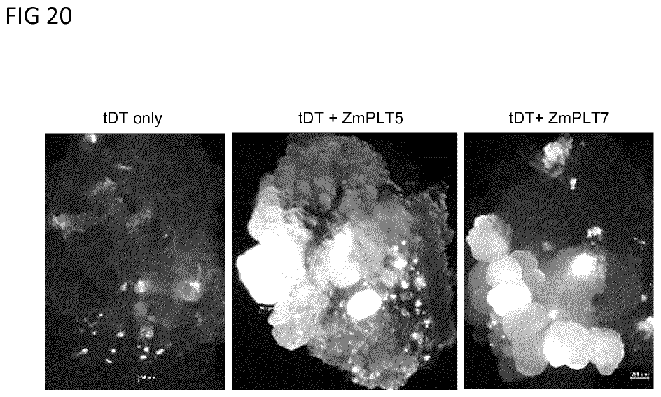

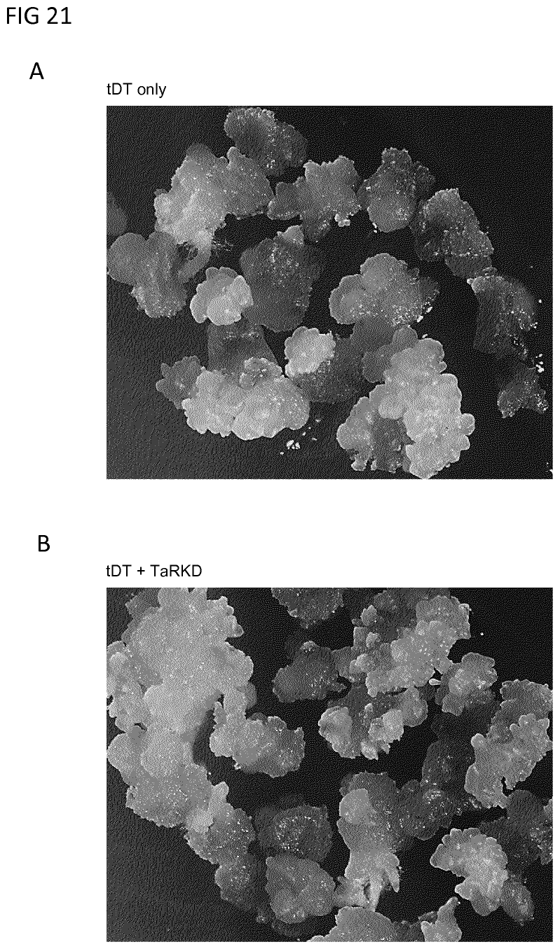

FIG. 2: Co-delivery of 15 ng TSA with construct pLH-Pat5077399-70Subi-tDt (FIG. 1) by microprojectile bombardment of 100 μg gold particles (size 0.6 μm).
A: Red fluorescence images showing tdTomato expressing cells in corn Hi II immature embryos 16 hours after bombardment (white spots). The images on the top are taken from control bombardments without TSA (No TSA), while the images on the bottom are taken from the co-bombardments with 15 ng of TSA.
B: Average numbers of red fluorescent cells per embryo 16 hours after the bombardment without (No TSA) or with 15 ng of TSA (15 ng TSA). Error bar=standard deviation.

FIG. 3: Co-delivery of different amounts of TSA (No TSA, 15 ng, 30 ng, and 45 ng) with construct pLH-Pat5077399-70Subi-tDt (FIG. 1) by microprojectile bombardment of 100 μg gold particles (size 0.6 μm) in Hi II immature embryos.
A: Average numbers of fluorescent cells per field in corn Hi II immature embryos 16 hours after bombardments with different amounts of TSA.
B: Percentage increase in average number of fluorescent cells when co-bombarded with different amounts of TSA. Error bar=standard deviation.

FIG. 4: pGEP359 construct map. tDT defines tdTomato gene. ZmLpCpf1 defines the maize codon-optimized CDS of the Lachnospiraceae bacterium CRISPR/Cpf1 (LbCpf1) gene.

FIG. 5: Co-delivery of 15 ng TSA with construct pGEP359 (FIG. 4) by microprojectile bombardment of 100 μg gold particles (size 0.6 μm).
A: Red fluorescence images showing tdTomato expressing cells in corn Hi II type II calluses 16 hours after bombardment. The images on the left were taken from control bombardments without TSA (No TSA), while the images on the right are taken from the co-bombardments with 15 ng of TSA.
B: Average numbers of red fluorescent cells per field 16 hours after bombarded without (No TSA) or with 15 ng of TSA. Error bar=standard deviation.

FIG. 6: Co-delivery of 15 ng TSA with construct pLH-Pat5077399-70Subi-tDt (FIG. 1) by microprojectile bombardment with 300 μg gold particles (0.6 μm).
A: Red fluorescence images showing tdTomato expressing cells in sugar beet friable calluses 24 hours after bombardment (white spots). The images on the top are taken from control bombardments without TSA (No TSA), while the images on the bottom show the co-bombardments with 15 ng TSA.
B: Average numbers of fluorescent cells per field 24 hours after bombarded without (−TSA) or with 15 ng of TSA (+TSA). Error bar=standard deviation.

FIG. 7: pGEP284 construct map. tDT defines tdTomato gene. TaCRISPR defines the wheat codon-optimized CDS of a CRISPR nuclease. sgGEP14 defines the guide RNA target to the first exon of maize glossy 2 gene.

FIG. 8: Co-delivery of different amounts of TSA (No TSA, 15 ng, 30 ng, and 45 ng) with gene-editing construct pGEP284 (FIG. 7) by microprojectile bombardment of 100 μg gold particles (size 0.6 μm). A: Site-specific InDel (insertion and deletion) rates in Hi II embryos 2 days after co-bombardment. B: Percentage changes in InDel rate when different amounts of TSA (No TSA 15 ng, 30 ng, and 45 ng, from left to right) were co-bombarded with a genome-editing construct pGEP284 in corn Hi II embryos.

FIG. 9: pGEP353 construct map. crGEP46 defines the crRNA46, which target to maize glycerate kinase gene (GLYK).

FIG. 10: Co-delivery of gene editing constructs pGEP359 (ZmLbCpf1, FIG. 4) and pGEP353 (crRNA46, FIG. 9) with 15 ng of TSA (on the right, 15 ng of TSA) or no TSA (on the left, No TSA) into corn Hi II callus.

FIG. 11: pGEP362 construct map. mNeonGreen defines mNeonGreen gene, which encodes the brightest monomeric green or yellow fluorescent protein with excitation maximum at 506 nm and emission maximum at 517 nm. ZmLpCpf1 defines the maize codon-optimized CDS of the Lachnospiraceae bacterium CRISPR/Cpf1 (LbCpf1) gene.

FIG. 12: Co-delivery of 250 ng 2,4-D with construct pGEP362 (FIG. 11) by microprojectile bombardment into corn Hi II immature embryos.
A: Green fluorescence images show mNeonGreen report gene expressing cells in corn Hi II immature embryos 16 hours after bombardment. The images on the top are taken from control bombardments without 2,4-D (No 2,4-D), while the images on the bottom show the co-bombardments with 250 ng of 2,4-D.
B: Average numbers of the green fluorescent cells per embryo 16 hours after the bombardment. Error bar=standard deviation.

FIG. 13: Co-delivery of different amounts of 2,4-D (0 ng, 125 ng, 250 ng, and 500 ng) with construct pGEP362 (FIG. 11) by microprojectile bombardment of 100 μg gold particles (size 0.6 μm).
A: Green fluorescence images showing mNeonGreen report gene expressing cells in corn Hi II type II callus cells 16 hours after co-bombarded with different amount of 2,4-D (0 ng, 125 ng, 250 ng, and 500 ng).
B: Average numbers of the green fluorescent cells per field 16 hours after the bombardment with different amount of 2,4-D (0 ng, 125 ng, 250 ng, and 500). Error bar=standard deviation.

FIG. 14: Co-delivery of 2,4-D with construct pGEP359 (FIG. 4) by microprojectile bombardment of 100 μg gold particles (size 0.6 μm) in leaves of corn plants (top: without 2,4-D, bottom: with 250 ng of 2,4-D) (exemplary tdT expression indicated by arrows).

FIG. 15: Co-delivery of 250 ng 6-BA or zeatin with construct pGEP359 (FIG. 4) by microprojectile bombardment with 100 μg of gold particle size (size 0.6 μm) in corn Hi II type II calluses.
A: red fluorescence images from left to right showing tdTomato report gene expressing cells in corn Hi II type II callus cells 16 hours after bombardment without hormone (no hormone), with 250 ng of 6-BA, or with 250 ng of zeatin.
B: Average numbers of the red fluorescent cells per field 16 hours after the bombardment. Error bar=standard deviation.

FIG. 16: pABM-BdEF1_ZmPLT5 construct map. Maize PLT5 gene (ZmPLT5) is driven by the strong constitutive EF1 promoter from Brachypodium (BDEF1).

FIG. 17: pABM-BdEF1_ZmPLT7 construct map. Maize PLT7 gene (ZmPLT7) is driven by the strong constitutive EF1 promoter from Brachypodium (BDEF1).

FIG. 18: pABM-BdEF1_TaRKD construct map. Wheat RKD gene (TaRKD) is driven by the strong constitutive EF1 promoter from Brachypodium (BDEF1).

FIG. 19: Co-delivery of 100 ng boost gene construct with construct pGEP359 (FIG. 4) by microprojectile bombardment with 100 μg of gold particle size (size 0.6 μm) into corn Hi II immature embryos.
A: red fluorescence images show tdTomato report gene expressing cells in corn Hi II immature embryos 16 hours after bombardment. The images on the left to right are taken from control bombardments without a boost (tDT only), or with the ZmPLT5 (FIG. 16) (tDT+ZmPLT5) or wheat RKD (TaRKD, FIG. 18) (tDT+TaRKD) boost construct.
B: Average numbers of the red fluorescent cells per embryo 16 hours after the bombardment. Error bar=standard deviation.

FIG. 20: tdTomato fluorescent embryogenic calluses were observed 12 days after co-bombarded with ZmPLT5 or ZmPLT7 gene construct. Figure shows red fluorescence images showing tdTomato report gene expressing in the embryogenic callus cells induced from the immature embryos 12 days after bombardment. Images from left to right showing the embryos bombarded with tDTomato report gene only (tDT only), or with 100 ng of boost ZmPLT5 (tDT+ZmPLT5), or ZmPLT7 gene construct (tDT+ZmPLT7)

FIG. 21: Callus induction in A188 immature embryos 17 days after co-bombardment of tdTomato with wheat RKD boost construct.
A: bright field image showing callus induction from the immature embryos bombarded with tDTomato report construct only.
B: bright field image showing callus induction from the immature embryos co-bombarded with tDTomato report and wheat RKD construct.

### EXAMPLES

Example 1: Co-Delivery of Trichostatin a (TSA) with a Construct Containing tdTomato Report Gene (i.e. pLH-Pat5077399-70Subi-tDt) by Microprojectile Bombardment Increased Transient Transformation Efficiency in Corn Immature Embryo without a TSA Pre-Treatment.

Procedure: Prepare corn immature embryo for bombardment: 8-10 days post pollination, maize ears (i.e. A188 or Hi II) with immature embryos size 0.8 to 1.8 mm were harvested. The ears were sterilized with 70% ethanol for 10-15 minutes. After a brief air-dry in a laminar hood, remove top ˜⅓ of the kernels from the ears with a shark scalpel, and pull the immature embryos out of the kernels carefully with a spatula. The fresh isolated embryos were placed onto the bombardment target area in an osmotic medium plate (see below) with scutellum-side up. Wrap the plates with parafilm and incubated them at 25° C. in dark for 4-20 hours before bombardment.

The amounts of TSA used for a bombardment with 100 μg of gold particles (approximately, 4.0-5.0×10⁷ 0.6 micron gold particles) are in range of 0.01 ng to 500 ng, preferred 0.1 to 50 ng. Plasmid DNA and TSA co-coating onto gold particles for bombardment: For 10 shots, 1 mg of gold particle size 0.6 micron (μm) in 50% (v/v) glycerol (100 μg gold particles per shot) in a total volume of 100 microliter (μl) was pipetted into a clear low-retention microcentrifuge tube. Sonicate for 15 seconds to suspend the gold particles. While vortex at a low speed, add the following in order to each 100 μl of gold particles:
Up to 10 μl of DNA (1.0 μg total DNA, 100 ng per shot)
100 μl of 2.5 M CaCl₂) (pre-cold on ice)
40 μl of 0.1 M cold spermidine

Close the lid and vortex the tube for 2-30 minutes at 0-10° C., and spin down the DNA-coated gold particles. After washing in 500 μl of 100% ethanol for two times, the pellet was resuspended in 120 μl of 100% ethanol. Finally, an appropriate amount of TSA (for a bombardment with 100 μg gold particles size 0.6 μm, TSA amount ranging from 0.01 to 500 ng, preferred 0.1-50 ng; TSA was dissolved in DMSO) was added into the re-suspended gold particle solution carefully. While vortexing at a low speed, pipet 10 μl of Plasmid DNA (pLH-Pat5077399-70Subi-tDt construct; FIG. 1) and TSA co-coated gold particles with a wide open 20 μl tip from the tube onto the center of the macrocarrier evenly since the particles tend to form clumps at this point, get the gold particles onto the macrocarriers as soon as possible. Air dry.

Bombardment was conducted using a Bio-Rad PDS-1000/He particle gun. The bombardment conditions are: 27-28 mm/Hg vacuum, 450 or 650 psi rupture disc, 6 mm gap distance, the specimen platform is in the second position from the bottom in the chamber at a distance of 60 mm. After bombardment the embryos were remained on the osmotic medium for another 16 hours, and then removed onto a type II callus induction medium plate (see below). 16-48 hours after bombardment, transient transformation was examined using a fluorescence microscope for the tdTomato gene expression at excitation maximum 554 nm and emission maximum 581 nm.

Type II callus induction medium: N6 salt, N6 vitamin, 1.0 mg/L of 2, 4-D, 100 mg/L of Caseine, 2.9 g/L of L-proline, 20 g/L sucrose, 5 g/L of glucose, 5 mg/L of AgNO3, 8 g/L of Bacto-agar, pH 5.8.

Osmotic medium: N6 vitamin, 1.0 mg/L of 2, 4-D, 100 mg/L of Caseine, 0.7 g/L of L-proline, 0.2 M Mannitol (36.4 g/L), 0.2 M sorbitol (36.4 g/L), 20 g/L sucrose, 15 g/L of Bacto-agar, pH 5.8.

In FIG. 2, the co-delivery of 15 ng TSA with construct pLH-Pat5077399-70Subi-tDt (FIG. 1) by microprojectile bombardment of 100 μg gold particles (size 0.6 μm) improves the DNA transient transformation in corn Hi II immature embryos. In FIG. 2A the red fluorescence images show tdTomato expressing cells in corn Hi II immature embryos 16 hours after bombardment with 15 ng TSA compared to control bombardments without TSA. The average number of the red fluorescent cells, i.e. positively transient transformed cells, per embryo 16 hours after the bombardment increased by 98.2% by co-delivery of 15 ng of TSA (FIG. 2B).

This co-delivery experiment has been repeated with different amounts of TSA—no TSA, 15 ng of TSA, 30 ng of TSA, and 45 ng of TSA (FIG. 3). The presence of TSA improves always the transient transformation in corn Hi II immature embryos. The average number of fluorescent cells, i.e. positively transient transformed cells, per field in corn Hi II immature embryos 16 hours showed an optimum around 30 ng of TSA (FIG. 3A). However even lower but also higher concentrations resulted in a significant increase of transient transformed cells (FIG. 3B).

Example 2: Co-Delivery of Trichostatin a (TSA) with a tdTomato Report Construct pGEP359 (FIG. 4) by Microprojectile Bombardment Promoted Transformation Efficiency in Corn Type II Callus without a TSA Pre-Treatment

Type II callus induction and selection: Hi II immature embryos size 0.8-1.8 mm were isolated as described in Example 1, and were placed onto type II callus induction medium (see below) immediately with scutellum-side up, in a density of 10-15 embryo per plate (diameter of 100 mm). Wrap the plates with parafilm, and culture the embryos in plate at 27° C. in the dark until type II callus emerged (˜2 weeks). Pick friable type II calluses under a stereoscope, and move them onto type II callus selection medium (see below). Repeat this process for 2-3 more times, and trash the embryos 4 weeks after induction. Select pre-embryo stage of type II callus under a stereoscope carefully based on: friability (highly friable), morphology (no embryo-like structure), color (fresh, white, semi-transparent). Select and subculture type II callus every 1-2 week in callus selection medium (see below) until the callus lines stabilized (about 3-5 rounds of selection). Stable type II callus lines were cultured in type II callus subculture medium (see below) every 1 to 2 weeks.

Preparation of type II callus for bombardment: Select and transfer highly friable type II callus at pre-embryo stage onto the bombardment target region in an osmotic medium plate (see Example 1) (single layer, no overlapping). Wrap the plates with parafilm and incubated at 25° C. in dark for 4-20 hours (preferred 4 hours) before bombardment.

Microprojectile bombardment and post-bombardment handlings were conducted using the same procedure as described in Example 1.

Type II callus induction medium: N6 salt, N6 vitamin, 1.0 mg/L of 2, 4-D, 100 mg/L of Caseine, 2.9 g/L of L-proline, 20 g/L sucrose, 5 g/L of glucose, 5 mg/L of AgNO3, 8 g/L of Bacto-agar, pH 5.8

Type II callus selection medium: N6 salt, N6 vitamin, 1.0 mg/L of 2, 4-D, 100 mg/L of Caseine, 2.9 g/L of L-proline, 20 g/L sucrose, 2 mg/L of AgNO3, 8 g/L of Bacto-agar, pH 5.8

Type II callus sub-culture medium: N6 salt, N6 vitamin, 1.0 mg/L of 2, 4-D, 100 mg/L of Caseine, 0.7 g/L of L-proline, 20 g/L sucrose, 8 g/L of Bacto-agar, pH 5.8

In FIG. 5, the co-delivery of 15 ng TSA with construct pGEP359 by microprojectile bombardment of 100 μg gold particles (size 0.6 μm) increased transient transformation in corn Hi II type II calluses. In FIG. 5A the red fluorescence images show tdTomato expressing cells in corn Hi II type II calluses 16 hours after bombardment with 15 ng TSA compared to control bombardments without TSA. The average number of fluorescent cells, i.e. positively transient transformed cells, per field in corn Hi II type II calluses 16 hours after bombardment increased by 43.3% by co-delivery of 15 ng of TSA (FIG. 5B).

Example 3: Co-Delivery of Trichostatin a (TSA) with Construct pLH-Pat5077399-70Subi-tDt by Microprojectile Bombardment Improved Transient Transformation in Sugar Beet Friable Callus

Sugar beet callus induction: young leaves from in vitro cultured sugar beet shoots in shoot culture medium (see below) were cut into small pieces (square, size 3-5 mm) in a laminar hood, and placed them onto callus induction medium (see below), in a density of 10-15 pieces per plate (diameter of 100 mm) with adaxial-side up. Wrap the plates with parafilm, and culture the leaf segments in plate at 23° C. in the dark for 6-8 weeks until callus emerged.

Preparation of sugar beet callus for bombardment: harvest friable fresh calluses under a stereoscope, and transfer them onto the bombardment target area in a sugar beet osmatic medium (see below) (single layer, no overlapping). Wrap the plates with parafilm and incubated at 25° C. in dark for 4-20 hours before bombardment.

Microprojectile bombardment and post-bombardment handlings were conducted using the same procedure descripted in Example 1, except for the amount of gold particles used for a bombardment was 300 μg.

Sugar beet shoot culture medium: MS, 0.25 mg/L of BAP, 30 g/L of sucrose, 8 g/I plant agar, pH 6.0

Sugar beet callus induction medium: MS, 2.0 mg/L of BAP, 15 g/L of sucrose, 8 g/I plant agar, pH 6.0

Sugar beet callus osmatic medium: MS, 2.0 mg/L of BAP, 15 g/L of sucrose, 0.2 M Mannitol (36.4 g/L), 0.2 M sorbitol (36.4 g/L), 8 g/I plant agar, pH 6.0

In FIG. 6, the co-delivery of 15 ng TSA with construct pLH-Pat5077399-70Subi-tDt (FIG. 1) by microprojectile bombardment with 300 μg gold particles (0.6 μm) improved transient transformation in sugar beet friable calluses. In FIG. 6A, the red fluorescence images show tdTomato expressing cells in sugar beet friable calluses 24 hours after bombardment with 15 ng TSA compared to control bombardments without TSA. The average number of fluorescent cells, i.e. positively transient transformed cells, per field 24 hours after bombardment increased by 193.7% by co-delivery of 15 ng of TSA (FIG. 6B).

Example 4: Co-delivery of trichostatin A (TSA) with gene editing constructs improved genome-editing efficiency in corn immature embryo.

Embryo isolation, microprojectile bombardment and post-bombardment handlings were performed using the same procedure as described in Example 1.

Two days after bombardment, the embryos were harvested and used for genomic DNA isolation use Plant DNA Isolation kit from Qiagen (Venlo, Netherlands). NGS (next generation sequencing) was conducted by Miseq platform of Illumina Inc. (San Diego, Calif., USA). InDel (insertion and deletion) rate was analyzed by means of CRISPResso (http://crispresso.rocks/).

In FIG. 8, the co-bombardment with TSA (No TSA, 15 ng, 30 ng, and 45 ng, from left to right) leads to an improved gene editing efficiency in Hi II embryos 2 days after bombardment. In FIG. 8A, the site-specific InDel rates in the Hi II embryos 2 days after co-bombardment with the gene editing construct pGEP284 (FIG. 7) for different amounts of TSA are shown, wherein the site-specific InDel rate indicates gene editing efficiency. The presence of TSA improves always the frequency of gene editing events in the corn Hi II immature embryos. The rates of InDel events, i.e. positively gene edited embryos, showed an optimum around 30 ng of TSA. However, even lower but also higher concentrations resulted in a significant increase of InDel rates compared to the absence of TSA. The percentage changes in InDel rate when different amounts of TSA were co-bombarded with a gene editing construct pGEP284 in corn Hi II embryos are shown in FIG. 8B).

Example 5: Co-Delivery of Trichostatin a (TSA) with Gene Editing Constructs pGEP359 (FIG. 4) and pGEP353 (FIG. 9) Improved Genome-Editing Efficiency in Corn Hi II Type II Calluses

Type II callus culture and microprojectile bombardment and post-bombardment handlings were performed using the same procedure as described in Example 2.

2-15 days after bombardment, the calluses were harvested and used for genomic DNA isolation with a Plant DNA Isolation kit from Qiagen. NGS (next generation sequencing) was conducted by Illumina Miseq platform. InDel (insertion and deletion) rate was analyzed by means of CRISPResso.

In FIG. 10, the co-bombardment of gene editing constructs pGEP359 (ZmLbCpf1, FIG. 4) and pGEP353 (crRNA46, FIG. 9) with 15 ng of TSA (on the right, 15 ng of TSA) or no TSA (on the left, No TSA) in corn Hi II calluses showed 13 days after co-bombardment an increase of the site-specific InDel (insertion and deletion) rate by factor 6.75 or 575%.

Example 6: Co-Delivery of Auxin 2,4-D with mNeonGreen Report Construct pGEP362 (FIG. 11) by Microprojectile Bombardment Increased its Transient Transformation Efficiency in Corn Immature Embryos

Embryo isolation and microprojectile bombardment and post-bombardment handlings were performed using the same procedure as described in Example 1.

The amounts of 2,4-D used for a bombardment with 100 μg of gold particles (approximately, 4.0-5.0×10⁷ 0.6 μm gold particles) are in range of 1.0 ng to 1000 ng, preferred 10 ng to 500 ng. Plasmid DNA and 2,4-D co-coating onto gold particles for bombardment were conducted as described in Example 1. 2,4-D stock solution (e.g. 1 mg/ml) is prepared in 100% DMSO.

In FIG. 12, the co-delivery of 250 ng 2,4-D with construct pGEP362 (FIG. 11) by microprojectile bombardment of 100 μg gold particles (size 0.6 μm) improves the DNA transient transformation in corn Hi II immature embryos. In FIG. 12A, the green fluorescence images show mNeonGreen report gene expressing cells in corn Hi II immature embryos 16 hours after bombardment. B: Average numbers of the green fluorescent cells per field 16 hours after the bombarded with 250 ng 2,4-D compared to control bombardments without 2,4-D. The co-bombardment with 250 ng of 2,4-D lead to an increase by 187% in the average number of the fluorescent cells per embryo (FIG. 12B).

Example 7: Co-Delivery of Auxin 2,4-D with mNeonGreen Report Construct pGEP362 (FIG. 11) by Microprojectile Bombardment Increased its Transient Transformation Efficiency in Corn Hi II Type II Calluses

Type II callus culture and microprojectile bombardment and post-bombardment handlings were performed using the same procedure as described in Example 2.

The amounts of 2,4-D used for a bombardment with 100 μg of gold particles (approximately, 4.0-5.0×10⁷ 0.6 μm gold particles) are in range of 1.0 ng to 1000 ng, preferred 10 ng to 500 ng. Plasmid DNA and 2,4-D co-coating onto gold particles for bombardment were conducted as described in Example 6.

In FIG. 13, the co-delivery of different amounts of 2,4-D (0 ng, 125 ng, 250 ng, and 500 ng) with construct pGEP362 (FIG. 11) by microprojectile bombardment of 100 μg gold particles (size 0.6 μm) improved the transient transformation in corn Hi II type II callus. The green fluorescence images showing mNeonGreen report gene expressing cells in corn Hi II type II callus cells 16 hours after co-bombarded with different amount of 2,4-D (2,4-D 0 ng, 125 ng, 250 ng, and 500 ng from top left to bottom right) shows a significant increase of fluorescence by the co-bombardment with 2,4-D (FIG. 13A). In FIG. 13B the average numbers of the green fluorescent cells per field 16 hours after the bombarded with different amount of 2,4-D (0 ng, 125 ng, 250 ng, and 500 ng) are shown. By the addition of 2,4-D the average number of the fluorescent cells have been increased by at least 34.8%.

Example 8: Co-Delivery of Auxin 2,4-D with tDTomato Report Construct pGEP359 (FIG. 4) by Microprojectile Bombardment Increased its Transient Transformation Efficiency in Leaves of Corn Plants

Corn plants have grown in greenhouse. In stage V8 microprojectile bombardment was conducted using a Bio-Rad PDS-1000/He particle gun. The bombardment conditions are: 27-28 mm/Hg vacuum, 450 or 650 psi rupture disc, 6 mm gap distance. 20 hours after bombardment, transient transformation was examined using a fluorescence microscope for the tdTomato gene expression at excitation maximum 554 nm and emission maximum 581 nm. Plasmid DNA and 2,4-D co-coating onto gold particles for bombardment were conducted as described in Example 1. 2,4-D stock solution (e.g. 25 mg/ml in DMSO).

In FIG. 14, the co-delivery of 2,4-D with construct pGEP359 (FIG. 4) by microprojectile bombardment improved the transient transformation in corn leaves.

Example 9: Co-Delivery of Cytokinins Like 6-BA or Zeatin with tDTomato Report Construct pGEP359 (FIG. 4) by Microprojectile Bombardment Increased its Transient Transformation Efficiency in Corn Hi II Type II Calluses

Type II callus culture and microprojectile bombardment and post-bombardment handlings were performed using the same procedure as described in Example 2.

The amounts of 6-BA or zeatin used for a bombardment with 100 μg of gold particles (approximately, 4.0-5.0×10⁷ 0.6 μm gold particles) are in range of 1.0 ng to 10000 ng, preferred 10 ng to 1000 ng. Plasmid DNA and the cytokinin co-coating onto gold particles for bombardment were conducted as described in Example 6.

In FIG. 15, the Co-delivery of 250 ng 6-BA or zeatin with construct pGEP359 (FIG. 4) by microprojectile bombardment with 100 μg of gold particle size 0.6 μm in corn Hi II type II calluses. The red fluorescence images showing tdTomato report gene expressing cells in corn Hi II type II callus cells 16 hours after bombardment (FIG. 15A), from left to right: control bombardment without hormone (no hormone), with 250 ng of 6-BA, and with 250 ng of zeatin. In FIG. 15B, the average numbers of the red fluorescent cells per field 16 hours after the bombardment are shown. 250 ng 6-BA co-bombardment led to a 35.8% increase and 250 ng zeatin a 31.2% increase in the average number of the fluorescent cells.

Example 10: Co-Delivery of a Boost Gene with the tDTomato Report Construct (FIG. 4) by Microprojectile Bombardment Increased its Transient Transformation Efficiency in Corn Immature Embryos

Embryo isolation, microprojectile bombardment and post-bombardment handlings were performed using the same procedure as described in Example 1.

Boost genes are co-bombarded with a fluorescent report construct (tdTomato gene, FIG. 4). The amounts of a boost gene construct (FIG. 16, FIG. 17, FIG. 18) used for a bombardment with 100 μg of gold particles (approximately, 4.0-5.0×10⁷ 0.6 μm gold particles) and 100 ng of the tDTomato report construct are in range of 10.0 ng to 1000 ng, preferred 50 ng to 100 ng. Plasmid DNA coating onto gold particles for bombardment were conducted as described in Example 1.

The boost effect is measured by its capability to increase the transient transformation frequency of the report gene 16-20 after bombardment of corn Hi II immature embryos.

In FIG. 19, the co-delivery of 100 ng of a boost gene construct with 100 ng of the tDTomato report construct (FIG. 4) by microprojectile bombardment of 100 μg gold particles (size 0.6 μm) improves the tDTomato gene transient transformation in corn Hi II immature embryos.

In FIG. 19A, the red fluorescence images show tDTomato report gene expressing cells in corn Hi II immature embryos 16 hours after bombardment. FIG. 19B: average numbers of the red fluorescent cells per embryo 16 hours after the bombarded with a boost gene construct compared to control bombardment with the report only (tDT only). The co-bombardment with 100 ng of ZmPLT5 boost gene construct (FIG. 16) (tDT+ZmPLT5) led to an increase by 102%, or with 100 ng of wheat RKD (TaRKD) (FIG. 18) (tDT+TaRKD) resulted into an increase by 144% in the average number of the fluorescent cells per embryo (FIG. 19B).

Example 11: Transient Over-Expression of Boost Genes Promote Transformation Frequency (TF)

Embryo isolation, microprojectile co-bombardment, and post-bombardment handlings were performed using the same procedure as described in Example 10. The boost effect on transformation is measured by its capability to increase the transformation frequency of the report gene at 12 days after bombardment of corn Hi II immature embryos (IE) without a selection.

As shown in Table 1, co-bombardment of tdTomato construct with ZmPLT5 led to an increase of 42.9% of the transformation frequency of tdTomato gene (over 16-fold increase compared to the control), while the co-bombardment with ZmPLT7 gave an increase of 53% of transformation frequency of tdTomato gene (over 16-fold increase compared to the control) 12 days after bombardment without a selection (FIG. 20).

TABLE 1
tDT transformation frequency (FT) at 12 days after
bombardment: FT is defined as the number of
embryos with at least one tDT expressing embryogenic
structures (No. of tDT positive IEs) from 100 embryos
bombarded.

| | tDT only | tDT + ZmPLT5 | tDT + ZMPLT7 |
|---|---|---|---|
| No. of tDT positive | 1/40 | 21/49 | 26/49 |
| IEs/total IEs | | | |
| tDT TF | 2.5% | 42.9% | 53.1% |

Example 12: Transient Over-Expression of Wheat RKD Boost Gene (SEQ ID NO: 6) Promote Callus Induction in A188 Immature Embryos

Embryo isolation, microprojectile co-bombardment, and post-bombardment handlings were performed using the same procedure as described in Example 10, and callus induction was conducted as described in Example 2.

Transient over-expression of wheat RKD gene led to a significant improvement in callus induction, the induction rate increased from 38% without TaRKD to 75% with 100 ng of TaRKD, nearly a doubling of the callus induction rate (FIG. 21).

1. A method for genetic modification in a plant cell comprising
(a) co-introducing into the plant cell
(i) a genome engineering component and
(ii) a second compound comprising
(ii.1) an epigenetically regulating chemical or an active derivative thereof, in particular a DNA methyltransferase inhibitor or a protein deacetylase inhibitor, preferably histone deacetylase inhibitor (HDACi), and/or
(ii.2) a phytohormone or an active derivative thereof, preferably selected from auxins, cytokinins and combinations thereof and/or
(ii.3) a protein causing improved plant regeneration from a somatic cell, a callus cell or embryonic cell or an expression cassette comprising a nucleic acid encoding said protein, and
(b) cultivating the plant cell under conditions allowing the genetic modification of the genome of said plant cell by activity of the genome engineering component in the presence of the second compound,
preferably wherein the genome engineering component (i) and/or the second compound (ii) is transiently active and/or transiently present in the plant cell.

2. The method of claim 1, wherein the genome engineering component comprises
a) a double-stranded DNA break (DSB) inducing enzyme or a nucleic acid encoding same, which preferably recognizes a predetermined site in the genome of said cell, and optionally a repair nucleic acid molecule, or
b) a single-stranded DNA or RNA break (SSB) inducing enzyme or a nucleic acid encoding same, which preferably recognizes a predetermined site in the genome of said cell, and optionally a repair nucleic acid molecule, or
c) a base editor enzyme, optionally fused to a disarmed DSB or SSB inducing enzyme, which preferably recognizes a predetermined site in the genome of said cell, or
d) an enzyme effecting DNA methylation, histone acetylation, histone methylation, histone ubiquitination, histone phosphorylation, histone sumoylation, histone ribosylation or histone citrullination, optionally fused to a disarmed DSB or SSB inducing enzyme, which preferably recognizes a predetermined site in the genome of said cell.

3. The method of claim 1, wherein the genome engineering component comprises an DSB or SSB inducing enzyme or a variant thereof selected from a CRISPR/Cas endonuclease, preferably a CRISPR/Cas9 endonuclease or a CRISPR/Cpf1 endonuclease, a zinc finger nuclease (ZFN), a homing endonuclease, a meganuclease and a TAL effector nuclease.

4. The method of claim 1, wherein transient activity of the genome engineering component in step b) comprises inducing one or more double-stranded breaks in the genome of the plant cell, one or more single strand breaks in the genome of the plant cell, one or more base editing events in the genome of the plant cell, or one or more DNA methylation, histone acetylation, histone methylation, histone ubiquitination, histone phosphorylation, histone sumoylation, histone ribosylation or histone citrullination in the genome of the plant cell.

5. The method of claim 4, wherein the induction of one or more double-stranded breaks or one or more single strand breaks is followed by non-homologous end joining (NHEJ) and/or by homology directed repair of the break(s) though a homologous recombination mechanism (HDR).

6. The method of claim 1, wherein in step b) the modification of said genome is selected from
a) a replacement of at least one nucleotide;
b) a deletion of at least one nucleotide;
c) an insertion of at least one nucleotide;
d) a change of the DNA methylation,
e) a change in histone acetylation, histone methylation, histone ubiquitination, histone phosphorylation, histone sumoylation, histone ribosylation or histone citrullination or
f) any combination of a)-e).

7. The method of claim 1, wherein the protein causing improved plant regeneration from a somatic cell, a callus cell or embryonic cell comprises an amino acid sequence which is selected from
a) a sequence as set forth in any of SEQ ID NO: 1, 3, 5, 7, 9, 11, 13, 15, 17, 19, 21, 23, 25, 27, 29 or 31,
b) a sequence having an identity of at least 60% to the sequence of (a),
c) a sequence encoded by a nucleic acid sequence as set forth in any of SEQ ID NO: 2, 4, 6, 8, 10, 12, 14, 16, 18, 20, 22, 24, 26, 28, 30 or 32, and
d) a sequence encoded by a nucleic acid sequence having an identity of at least 60% to the nucleic acid sequence of (c).

8. The method of claim 1, further comprising a step of pretreatment of the plant cell to be used in step (a), said pretreatment comprising culturing the plant cell or plant material comprising same in a medium containing the epigenetically regulating chemicals or an active derivative thereof, the phytohormone or the active derivative thereof, the protein causing improved plant regeneration, or any combination thereof.

9. A genetically modified plant cell obtained or obtainable according to the method of claim 1.

10. A plant or a plant part comprising the genetically modified plant cell of claim 9.

11. A microparticle coated with at least
(i) a genome engineering component and
(ii) a second compound comprising
(ii.1) an epigenetically regulating chemical or an active derivative thereof, in particular a DNA methyltransferase inhibitor or a protein deacetylase inhibitor, preferably histone deacetylase inhibitor (HDACi), and/or
(ii.2) a phytohormone or an active derivative thereof, preferably selected from auxins, cytokinins and combinations thereof and/or
(ii.3) a protein causing improved plant regeneration from a somatic cell, a callus cell or embryonic cell or an expression cassette comprising a nucleic acid encoding said protein.

12. A kit for the genetic modification of a plant genome by microprojectile bombardment, comprising
(i) one or more microparticles, and
(ii) means for coating the microparticles with at least a genome engineering component and a second compound comprising
(1) an epigenetically regulating chemical or an active derivative thereof, in particular a DNA methyltransferase inhibitor or a protein deacetylase inhibitor, preferably histone deacetylase inhibitor (HDACi), and/or
(2) a phytohormone or an active derivative thereof, preferably selected from auxins, cytokinins and combinations thereof and/or
(3) a protein causing improved plant regeneration from a somatic cell, a callus cell or embryonic cell or an expression cassette comprising a nucleic acid encoding said protein.

13. A method for producing a genetically modified plant, comprising the steps:
(a) genetically modifying a plant cell according to the method of claim 1, and
(b) regenerating a plant from the modified plant cell of step (a),
preferably wherein the produced plant does not contain any of the genome engineering component and the second compound, co-introduced in step a).

14. A genetically modified plant or a part thereof obtained or obtainable by the method of claim 13, or a progeny plant thereof.

15. The method of claim 1, comprising the use of an epigenetically regulating chemical or an active derivative thereof, in particular a DNA methyltransferase inhibitor or a protein deacetylase inhibitor, preferably histone deacetylase inhibitor (HDACi), and/or a phytohormone or an active derivative thereof, preferably selected from auxins, cytokinins and combinations thereof, and/or a protein causing improved plant regeneration from a somatic cell, a callus cell or embryonic cell or an expression cassette comprising a nucleic acid encoding said protein.
